(19) United States
(12) Patent Application Publication
(10) Pub. No.: US 2016/0368993 A1
(43) Pub. Date: Dec. 22, 2016

(54) ALPHA-V BETA-6 INTEGRIN-BINDING ANTIBODY FRAGMENTS

(71) Applicant: THE REGENTS OF THE UNIVERSITY OF CALIFORNIA, Oakland, CA (US)

(72) Inventors: Julie L. Sutcliffe, Davis, CA (US); JASON BRADLEY WHITE, Davis, CA (US)

(73) Assignee: THE REGENTS OF THE UNIVERSITY OF CALIFORNIA, Oakland, CA (US)

(21) Appl. No.: 15/171,952

(22) Filed: Jun. 2, 2016

Related U.S. Application Data
(63) Continuation of application No. PCT/US2014/068806, filed on Dec. 5, 2014.
(60) Provisional application No. 61/913,219, filed on Dec. 6, 2013.

(51) Int. Cl. C07K 16/28; A61K 51/10

(52) U.S. Cl. CPC C07K 16/2839; A61K 51/1027; C07K 2317/35; C07K 2317/622; C07K 2317/56

(57) ABSTRACT

The present invention provides isolated, engineered multivalent antibody fragments that specifically bind α_{V}β₆ integrin. In particular embodiments, the antibody fragments of the present invention further comprise an imaging agent and/or a therapeutic agent. In some instances, the antibody fragments also include unnatural amino acids and/or inserted cysteine or thiol residues at the C-terminus of the scFv molecules of the fragments. The anti-α_{V}β₆ integrin antibody fragments provided herein are particularly useful for imaging a tumor, organ or tissue and for treating an α_{V}β₆ integrin-mediated disease or disorder. Compositions and kits containing the multivalent antibody fragments of the present invention are also described herein.

### CROSS-REFERENCES TO RELATED APPLICATIONS

The present application is a continuation of PCT/US2014/068806, filed Dec. 5, 2014, which application claims priority to U.S. Provisional Application No. 61/913,219, filed Dec. 6, 2013, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. DE-SC0002061 and DE-SC0008385, awarded by the Department of Energy. The Government has certain rights in this invention.

### BACKGROUND OF THE INVENTION

Integrins are a large family of cell-surface receptors responsible for mediating cell-cell and cell-extracellular matrix (ECM) adhesion. There are at least 24 different integrins, each a heterodimer composed of an α and β subunit, whose expression is determined by several factors including tissue type, stage of development, and various tissue pathologies such as inflammation and cancer. Although they do not possess any intrinsic enzymatic activity, subsequent to ligand binding, integrins translate extracellular cues into intracellular signals by bringing into juxtaposition a complex of cytoplasmic structural and signaling molecules that then interact and determine the cellular response. As integrins are involved in most elements of cell behavior including motility, proliferation, invasion, and survival, their roles in disease have been widely reported. It has become clear that the αᵥβ₆ integrin is a major new target in cancer. Although αᵥβ₆ integrin is epithelial-specific, it is weak or undetectable in most resting epithelial tissues but is strongly upregulated in many types of cancer, often at the invasive front.

The overexpression of the αᵥβ₆ integrin has been demonstrated to play a role in the invasive and metastatic characteristics of several cancers including oral squamous cell carcinoma, pancreatic, ovarian, cervical, and most recently was found to be overexpressed in breast ductal carcinoma in situ (DCIS). Increased expression of αᵥβ₆ also correlates with increase with advancing tumor grade; demonstrating that this integrin can serve as a prognostic indicator and an attractive target for molecular imaging. Consequently, the development of novel molecular imaging agents to target αᵥβ₆ in vivo could significantly improve the diagnosis and treatment of these diseases. Antibody-based imaging techniques with positron emission tomography (PET), which combine the inherent specificity of antibodies with the high sensitivity of PET, have therefore emerged as valuable complements to their therapeutic counterparts.

Antibodies provide excellent targeting characteristics including high specificity and high affinity; however, these reagents possess limited applications in diagnostic molecular imaging due to their large size, slow blood clearance, and associated effector functions. In clinical PET imaging, these properties are undesirable and contribute to increased circulation times, which prevents high-contrast tumor images from being rapidly obtained. In addition, the large size of intact antibodies inhibits effective tumor penetration.

Recombinant protein engineering has made it possible to dissect antibodies in order to eliminate undesirable features and reassemble them into engineered antibody fragments while preserving high specificity to the target antigen. See, FIG. 1. Antibody fragments are smaller in size than their intact counterparts, but exhibit rapid tumor uptake and shorter blood circulation times.

The smallest antibody fragment that retains the minimal binding regions necessary for antigen recognition is the Fv module, which is comprised of single V_{H} and V_{L} chains held together by non-covalent interactions. However these small chains are unstable and often form aggregates in vivo.

Single-chain Fv (scFv) fragments consist of V_{H} and V_{L} chains connected by a flexible peptide linker. The most commonly used linker consists of a combination of serine and glycine residues (Gly4Ser)ₙ that provide flexibility and hydrophilicity as well as protease resistance. Connection of the V_{H} and V_{L} chains by linkers containing more than 12 amino acid residues provides enough flexibility for the two chains to assemble into the natural Fv orientation that contains the antigen binding site. The entire scFv construct can be encoded by a single gene and expressed in bacterial or mammalian expression systems, giving these fragments an added advantage over their intact counterparts. The compact size of scFv fragments (˜25 kDa) allows rapid accumulation in tumors, but their clinical applications remain limited and at times unfeasible because of very rapid clearance and monovalent binding.

In view of the foregoing, there is a need in the art for tumor targeting agents which not only provide high tumor specificity and selectivity for αᵥβ₆-expressing cells and tumors, but are also metabolically stable and retained in the αᵥβ₆-expressing cells and tumors. The present invention satisfies this need and provides related advantages as well.

### BRIEF SUMMARY OF THE INVENTION

In some aspects, the present invention provides an isolated multivalent antibody fragment that specifically binds αᵥβ₆ integrin comprising two or more single-chain Fv (scFv) molecules that associate with each other, wherein each scFv molecule independently comprises the following structure: (a) a light chain variable (V_{L}) region and a heavy chain variable (V_{H}) region of an antibody that specifically binds αᵥβ₆ integrin; and (b) a peptide linker between the V_{L} region and the V_{H} region.

In particular embodiments, the antibody fragment comprises at least one unnatural amino acid (e.g., at least 1, 2, 3, 4, 5, 6, 7, 8, 9, 10 or more unnatural amino acids in one, two, or more of the scFv molecules or in each scFv molecule). In certain instances, the at least one unnatural amino acid has been inserted at a prescribed location and/or at multiple positions in one, two, or more of the scFv molecules or in each scFv molecule. In some embodiments, a cysteine or thiol residue has been inserted at the C-terminus of one, two, or more of the scFv molecules or of each scFv molecule.

In some embodiments, the V_{H} region and the V_{L} region are derived from the humanized hu6.3G9 antibody. The V_{H} region can include a polypeptide having the amino acid sequence of SEQ ID NO: 1. In some embodiments, the V_{H} region comprises a polypeptide having at least 90%, 91%, 92%, 93%, 94%, 95%, 96%, 97%, 98%, 99%, or 100% identity to SEQ ID NO:1. The V_{L} region can include a polypeptide having the amino acid sequence of SEQ ID NO:2. In some embodiments, the V_{L} region comprises a polypeptide having at least 90%, 91%, 92%, 93%, 94%, 95%, 96%, 97%, 98%, 99%, or 100% identity to SEQ ID NO:2.

In some embodiments, the peptide linker is less than about 15 amino acids in length. In particular embodiments, the peptide linker is from 0 to about 8 amino acids in length.

In other embodiments, the antibody fragment is selected from the group consisting of a diabody, a minibody, a triabody, a tetrabody, and a combination thereof. In particular embodiments, the antibody fragment comprises two, three, or four scFv molecules that associate with each other to form a diabody, a minibody, a triabody, or a tetrabody.

In some embodiments, an imaging agent or a therapeutic agent is conjugated to the antibody fragment, i.e., to form a conjugate between the antibody fragment and the agent. The imaging agent or the therapeutic agent can be conjugated to a predetermined site in the antibody fragment (e.g., the agent can be conjugated to a predetermined site in one, two, or more of the scFv molecules or in each scFv molecule). Optionally, the predetermined site is an unnatural amino acid in the antibody fragment (e.g., the predetermined site is an unnatural amino acid in one, two, or more of the scFv molecules or in each scFv molecule). In some instances, the predetermined site is a cysteine or thiol residue that has been inserted at the C-terminus of one, two, or more of the scFv molecules or of each scFv molecule. In some embodiments, the radionuclide is selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I.

Also, provided herein is a composition comprising an antibody fragment described herein (e.g., a conjugate) and a pharmaceutically acceptable carrier. Further, the present invention provides a kit for imaging (e.g., in vivo imaging) or therapy, the kit comprising an antibody fragment described herein (e.g., a conjugate) or a composition thereof, and instructions for use thereof for imaging or therapy.

In other aspects, the present invention provides a method of in vivo imaging of a target tissue in a subject, the method comprising:
(a) administering to the subject in need of such imaging, an antibody fragment described herein or a composition thereof, wherein an imaging agent is conjugated to the antibody fragment; and
(b) detecting the antibody fragment to determine where the antibody fragment is concentrated in the subject.

In some embodiments, the target tissue is a cancerous tissue or an organ. The cancerous tissue may be associated with pancreatic cancer, breast cancer, colorectal cancer, prostate cancer, or oral squamous cell carcinoma.

In some embodiments, the imaging agent is a radionuclide, and radiation from the radionuclide is used to determine where the antibody fragment is concentrated in the subject.

The radionuclide may be selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I. In some instances, the antibody fragment is detected by Magnetic Resonance Imaging (MRI), Magnetic Resonance Spectroscopy (MRS), Single Photon Emission Computerized Tomography (SPECT), Positron Emission Tomography (PET), or optical imaging.

In some embodiments, the subject has an αᵥβ₆ integrin-mediated disease or disorder.

In yet other aspects, provided herein is a method of treating an αᵥβ₆ integrin-mediated disease or disorder in a subject in need thereof, the method comprising administering to the subject a therapeutically effective amount of an antibody fragment described herein or a composition thereof, wherein a therapeutic agent is conjugated to the antibody fragment. In some instances, the antibody fragment specifically binds αᵥβ₆ integrin.

In some embodiments, the therapeutic agent is a radionuclide. The radionuclide may be selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I. In some embodiments, the αᵥβ₆ integrin-mediated disease or disorder is pancreatic cancer, breast cancer, colorectal cancer, prostate cancer, or oral squamous cell carcinoma. In particular embodiments, the therapeutically effective amount of the antibody fragment is an amount sufficient to target delivery of the therapeutic agent to a cell expressing the αᵥβ₆ integrin.

In certain embodiments, the present invention provides an isolated and/or purified multivalent antibody fragment (e.g., diabody) specific for αᵥβ₆ integrin comprising one or more unnatural amino acids (UAAs) that are incorporated into one, two, or more of the scFv molecules or into each scFv molecule of the antibody fragment. In some instances, the incorporation of UAAs into the antibody fragment enables site-specific labeling. In other instances, the incorporation of UAAs into regions that would impair antibody fragment affinity is avoided. In other instances, the incorporation of UAAs allows for labeling functionality beyond what is afforded by the 21 canonical amino acids. In other embodiments, the present invention provides an isolated and/or purified multivalent antibody fragment (e.g., diabody) specific for αᵥβ₆ integrin comprising one or more exposed cysteine residues that are incorporated into one, two, or more of the scFv molecules or into each scFv molecule of the antibody fragment. In some instances, the incorporation of exposed cysteine residues allows for covalent bonding between heavy and light chains of the antibody fragment, thereby promoting stability. In other instances, the incorporation of exposed cysteine residues (e.g., inserted at the C-terminus of each scFv molecule) enables site-specific labeling via selective thiol chemistry, avoiding incorporation of labels at sites that are important for integrin binding. In yet other embodiments, the present invention provides an isolated and/or purified multivalent antibody fragment (e.g., diabody) specific for αᵥβ₆ integrin comprising one or more UAAs and one or more exposed cysteine residues that are incorporated into one, two, or more of the scFv molecules or into each scFv molecule of the antibody fragment.

In particular aspects, the present invention provides a composition comprising one or a combination of conjugates, wherein each conjugate independently comprises:
(a) an antibody fragment described herein; and
(b) an imaging agent or a therapeutic agent covalently bound to the antibody fragment, wherein at least one cysteine or thiol residue is inserted in the antibody fragment, and/or wherein at least one unnatural amino acid is inserted into the antibody fragment.

In some embodiments, the antibody fragment binds specifically to αᵥβ₆ integrin. In other embodiments, the antibody fragment comprises one or more unnatural amino acids (e.g., 1, 2, 3, 4, 5, 6, 7, 8, 9, 10 or more unnatural amino acids) that have been inserted at prescribed locations into one, two, or more of the scFv molecules or into each scFv molecule of the antibody fragment. In such instances, the unnatural amino acids can be the same or different and/or can be inserted at multiple positions (e.g., at 2, 3, 4, 5, 6, 7, 8, 9, 10 or more positions) in one, two, or more of the scFv molecules or in each scFv molecule of the antibody fragment. In yet other embodiments, the antibody fragment comprises one or more exposed cysteine residues (e.g., 1, 2, 3, 4, 5, 6, 7, 8, 9, 10 or more cysteine residues) that have been inserted at prescribed locations into one, two, or more of the scFv molecules or into each scFv molecule of the antibody fragment (e.g., at the C-terminus of each scFv molecule). In particular embodiments, both unnatural amino acids and exposed cysteine residues have been inserted at prescribed locations into one, two, or more of the scFv molecules or into each scFv molecule of the antibody fragment. Non-limiting examples of antibody fragments include a diabody, a minibody, a triabody, a tetrabody, and combinations thereof. In certain instances, the imaging agent or the therapeutic agent can be the same or different for each conjugate.

Other objects, features, and advantages of the present invention will be apparent to one of skill in the art from the following detailed description and figures.

### BRIEF DESCRIPTION OF THE DRAWINGS

FIG. 1 provides a comparison of the structure, size and clearance rates of antibody fragments.

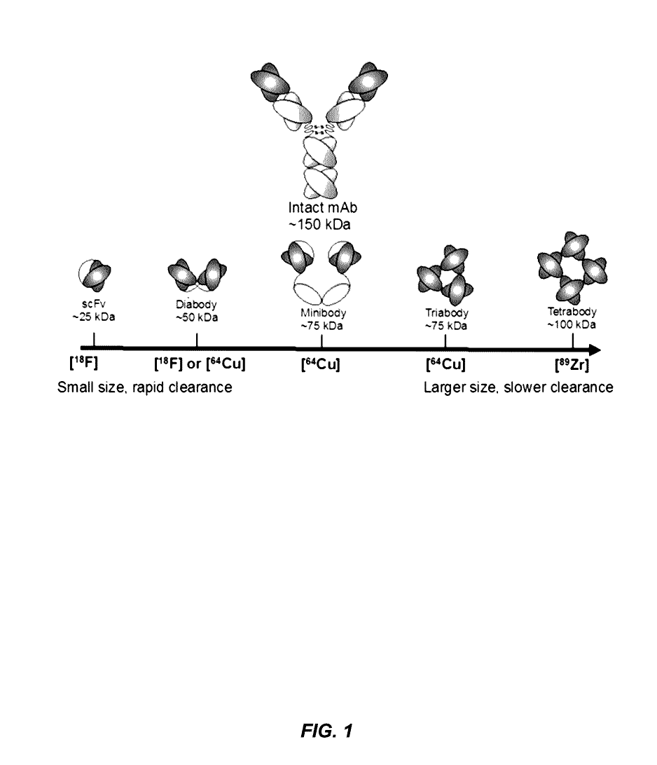

FIG. 2 shows a schematic representation of the effects of linker lengths on antibody fragment assemblies.

FIG. 3 shows the amino acid and DNA sequence of the variable heavy chain (V_{H}) of an exemplary anti-αᵥβ₆ diabody. SEQ ID NO:1 represents the amino acid sequence and SEQ ID NO:3 refers to the nucleic acid sequence.

FIG. 4 shows the amino acid and DNA sequence of the variable light chain (V_{L}) of an exemplary anti-αᵥβ₆ diabody. SEQ ID NO:2 represents the amino acid sequence and SEQ ID NO:4 refers to the nucleic acid sequence.

FIGS. 5A-B show schematic representations of an anti-αᵥβ₆ diabody and an anti-αᵥβ₆ Cys-diabody. FIG. 5A shows an anti-αᵥβ₆ diabody that includes V_{H} and V_{L} domains that are joined using a three amino acid linker (GGS) to induce dimerization. FIG. 5B shows an anti-anti-αᵥβ₆ Cys-diabody that includes V_{H} and V_{L} domains that are joined using a three amino acid linker (GGS) to induce dimerization, and the cysteine codon GGC at the C-terminus of the polypeptide. L=leader (secretion signal) peptide; 6×His=Histidine tag (purification tag).

FIGS. 6A-D show the purity for the engineered anti-αᵥβ₆ diabody and anti-αᵥβ₆ cys-diabody. FIGS. 6A and 6C show SDS-PAGE of purified anti-αᵥβ₆ diabody (FIG. 6A) and anti-αᵥβ₆ Cys-diabody (FIG. 6C). FIGS. 6B and 6D show size exclusion chromatography (SEC) of purified anti-αᵥβ₆ diabody (FIG. 6B) and purified anti-αᵥβ₆ Cys-diabody (FIG. 6D). The retention time of the purified anti-αᵥβ₆ diabody and the anti-αᵥβ₆ Cys-diabody was 23.2 minutes which is later than the carbonic anhydrase control (T_{R}=22.4 min).

FIGS. 7A-C show the synthesis of [¹⁸F]-SFB (FIG. 7A) and radiolabeling of the anti-αᵥβ₆ diabody (FIG. 7B) and the anti-αᵥβ₆ Cys-diabody (FIG. 7C) on ε-amines of exposed lysine residues with [¹⁸F]-SFB.

FIG. 8 shows the conjugation of NOTA-malemide to the reduced anti-αᵥβ₆ Cys-diabody and subsequent site-specific labeling with [⁶⁴Cu].

FIG. 9 shows competitive binding ELISA results between the anti-αᵥβ₆ diabody and fibronectin for binding to immobilized αᵥβ₆ integrin. Fibronectin is a ligand for αᵥβ₆. Each point represents the average of triplicate experiments with error bars representing the standard deviation.

FIG. 10 shows competitive binding ELISA results between the anti-αᵥβ₆ Cys diabody and fibronectin for binding to immobilized αᵥβ₆ integrin. Fibronectin is a ligand for αᵥβ₆. Each point represents the average of triplicate experiments with error bars representing the standard deviation.

FIGS. 11A-D show flow cytometry results for anti-αᵥβ₆ diabody (FIGS. 11A and 11B) and anti-αᵥβ₆ Cys-diabody (FIGS. 11C and 11D) in αᵥβ₆-negative cells (Dx3Puro cells; FIGS. 11A and 11C) and αᵥβ₆-positive cells (Dx3Puroβ₆ cells; FIGS. 11B and 11D). No fluorescent shift (A₄₈₈) was observed from the Dx3Puro cell lines after incubation with the IgG2a isotype control, the 10D5 positive control, or the anti-αᵥβ₆ diabody (FIG. 11A). A shift in fluorescence (A₄₈₈) was only observed from the Dx3Puroβ₆ cell line after incubation with the 10D5 positive control and the anti-αᵥβ₆ diabody (FIG. 11B). Similar results were seen for the anti-αᵥβ₆ Cys-diabody in the cell lines.

FIG. 12 shows the immunoreactivity of the [¹⁸F]-SFB-αᵥβ₆ diabody and the [¹⁸F]-SFB-αᵥβ₆ Cys-diabody after incubation with αᵥβ₆-negative cells (Dx3Puro cells) and αᵥβ₆-positive cells (Dx3Puroβ₆ cells).

FIG. 13 shows the immunoreactivity of the [⁶⁴Cu]-NOTA-αᵥβ₆ Cys-diabody after incubation with αᵥβ₆-negative cells (Dx3Puro cells) and αᵥβ₆-positive cells (Dx3Puroβ₆ cells).

### DETAILED DESCRIPTION OF THE INVENTION

### I. Introduction

The present invention is based, in part, upon the surprising discovery that engineered, multivalent antibody fragments (e.g., diabodies, minibodies, triabodies, and tetrabodies) that are based on two or more scFv chains of a humanized anti-αᵥβ₆ integrin antibody can be used as an in vivo imaging agent and/or therapeutic agent to target αᵥβ₆-expressing cells or tumors in a subject, e.g., a human subject. In particular, described herein are exemplary radiolabeled anti-αᵥβ₆ diabodies (e.g., [¹⁸F]-SFB-αᵥβ₆ diabodies) and variants thereof (e.g., [¹⁸F]-SFB-αᵥβ₆ Cys-diabodies, [⁶⁴Cu]-NOTA-αᵥβ₆ Cys-diabodies) that possess advantageous targeting characteristics and in vivo pharmacokinetics.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein to modify a numerical value indicates a defined range around that value. As a non-limiting example, if “X” were the value, “about X” would indicate a value from 0.9X to 1.1X, and more preferably, a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

When the term “about” is applied to describe the beginning of a numerical range, it applies to both ends of the range. Thus, “from about 700 to 850 nm” is equivalent to “from about 700 nm to about 850 nm.” When “about” is applied to describe the first value of a set of values, it applies to all values in that set. Thus, “about 680, 700, or 750 nm” is equivalent to “about 680 nm, about 700 nm, or about 750 nm.” However, when the term “about” is applied to describe only the end of the range or only a later value in the set of values, it applies only to that value or that end of the range. Thus, the range “about 2 to about 10” is the same as “about 2 to 10,” but the range “2 to about 10” is not.

The term “about” can also indicate an acceptable degree of error for the quantity measured given the nature or precision of the measurements. Typical, exemplary degrees of error are within 20%, preferably within 10%, and more preferably within 5% of a given value or range of values. Numerical quantities given herein are approximate unless stated otherwise, meaning that the term “about” can be inferred when not expressly stated.

The term “antibody” includes large (150 kDa) “Y-shaped” molecules that consist of two identical light chains (˜220 amino acids) and two identical heavy chains (˜440 amino acids) that are held together by a combination of covalent (disulfide) and non-covalent interactions. Each light and heavy chain consists of repeating segments of constant or variable regions that contain one intrachain disulfide bond. The variable regions are located at the N-termini of the light and heavy chains, while the constant domains are located at the C-termini of the light and heavy chains. The N-termini of the light and heavy chains come together to form the antigen-binding site. The light chain is comprised of one variable domain and one constant domain and the heavy chain is comprised of one variable domain and three constant domains. Located at the ends of the “Y” are two identical (bivalent) antigen-binding sites. The distance between the two antigen binding sites varies due to the flexible hinge region, and as a result, the antigen binding efficiency can be greatly increased. The formation of the antigen-binding region is caused by the pairing of the variable region from the heavy chain (V_{H}) with the variable region of the light chain (V_{L}). Variations in amino acid sequences of the variable regions are responsible for the vast diversity of antigen-binding sites, and the greatest variability occurs throughout three hypervariable regions, termed complementary determining regions (CDRs). The tail region of the antibody, known as the F_{C} region, is comprised of two constant domains (C_{H}2, and C_{H}3) from each of the heavy chains. The F_{C} region is responsible for recruiting effector functions through binding of F_{C} receptors on neutrophils and macrophages.

The term “single chain Fv” or “scFv” refers to an antibody fragment including the V_{H} and V_{L} domains of a single arm of an antibody joined by a linker such that the V_{H} and V_{L} domains pair to form a monovalent molecule, or the V_{H} domain of one scFv molecule pairs with (e.g., associates with or specifically binds to) the V_{L} domain of another scFv molecule to form multivalent structures (e.g., diabodies, triabodies, tetrabodies, minibodies, etc.).

The term “humanized antibody” refers to an antibody comprising at least one chain comprising variable region framework residues substantially from a human antibody chain (referred to as the acceptor immunoglobulin or antibody) and at least one CDR substantially from a mouse antibody, (referred to as the donor immunoglobulin or antibody). See, e.g., Queen et al., Proc. Natl. Acad. Sci. USA 86: 10029 10033 (1989), U.S. Pat. No. 5,530,101, U.S. Pat. No. 5,585,089, U.S. Pat. No. 5,693,761, WO 90/07861, and U.S. Pat. No. 5,225,539. The constant region(s), if present, can also be substantially or entirely from a human immunoglobulin. Methods of making humanized antibodies are known in the art. See, e.g., U.S. Pat. No. 7,256,273.

The term “amino acid” refers to naturally-occurring and unnatural amino acids, as well as amino acid analogs and amino acid mimetics that function in a manner similar to the naturally occurring amino acids.

Amino acids may be referred to herein by either their name, their commonly known three letter symbols or by the one-letter symbols recommended by the IUPAC-IUB Biochemical Nomenclature Commission. Additionally, nucleotides may be referred to by their commonly accepted single-letter codes.

Naturally-occurring amino acids are those encoded by the genetic code, as well as those amino acids that are later modified, e.g., γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. Other examples of naturally-occurring amino acids include pyrolysine and selenocysteine.

Unnatural amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an α carbon that is bound to a hydrogen, a carboxyl group, an amino group, but have modified R (i.e., side-chain) groups. Non-limiting examples of amino acid analogs include homoserine, norleucine, methionine sulfoxide, and methionine methyl sulfonium.

Non-limiting examples of unnatural amino acids include 1-aminocyclopentane-1-carboxylic acid (Acp), 1-aminocyclobutane-1-carboxylic acid (Acb), 1-aminocyclopropane-1-carboxylic acid (Acpc), citrulline (Cit), homocitrulline (HoCit), α-aminohexanedioic acid (Aad), 3-(4-pyridyl)alanine (4-Pal), 3-(3-pyridyl)alanine (3-Pal), propargylglycine (Pra), α-aminoisobutyric acid (Aib), α-aminobutyric acid (Abu), norvaline (Nva), α,β-diaminopropionic acid (Dpr), α,γ-diaminobutyric acid (Dbu), α-tert-butylglycine (Bug), 3,5-dinitrotyrosine (Tyr(3,5-di NO₂)), norleucine (Nle), 3-(2-naphthyl)alanine (Nal-2), 3-(1-naphthyl)alanine (Nal-1), cyclohexylalanine (Cha), di-n-propylglycine (Dpg), cyclopropylalanine (Cpa), homoleucine (Hle), homoserine (HoSer), homoarginine (Har), homocysteine (Hcy), methionine sulfoxide (Met(O)), methionine methylsulfonium (Met (S-Me)), α-cyclohexylglycine (Chg), 3-benzo-thienylalanine (Bta), taurine (Tau), hydroxyproline (Hyp), O-benzyl-hydroxyproline (Hyp(Bzl)), homoproline (HoPro), β-homoproline WHoPro), thiazolidine-4-carboxylic acid (Thz), nipecotic acid (Nip), isonipecotic acid (IsoNip), 3-carboxymethyl-1-phenyl-1,3,8-triazaspiro[4,5]decan-4-one (Cptd), tetrahydro-isoquinoline-3-carboxylic acid (3-Tic), 5H-thiazolo[3,2-a]pyridine-3-carboxylic acid (Btd), 3-aminobenzoic acid (3-Abz), 3-(2-thienyl)alanine (2-Thi), 3-(3-thienyl)alanine (3-Thi), α-aminooctanedioc acid (Asu), diethylglycine (Deg), 4-amino-4-carboxy-1,1-dioxo-tetrahydrothiopyran (Acdt), 1-amino-1-(4-hydroxycyclohexyl) carboxylic acid (Ahch), 1-amino-1-(4-ketocyclohexyl)carboxylic acid (Akch), 4-amino-4-carboxytetrahydropyran (Actp), 3-nitrotyrosine (Tyr(3-NO₂)), 1-amino-1-cyclohexane carboxylic acid (Ach), 1-amino-1-(3-piperidinyl)carboxylic acid (3-Apc), 1-amino-1-(4-piperidinyl)carboxylic acid (4-Apc), 2-amino-3-(4-piperidinyl) propionic acid (4-App), 2-aminoindane-2-carboxylic acid (Aic), 2-amino-2-naphthylacetic acid (Ana), (2S,5R)-5-phenylpyrrolidine-2-carboxylic acid (Ppca), 4-thiazoylalanine (Tha), 2-aminooctanoic acid (Aoa), 2-aminoheptanoic acid (Aha), ornithine (Orn), azetidine-2-carboxylic acid (Aca), α-amino-3-chloro-4,5-dihydro-5-isoazoleacetic acid (Acdi), thiazolidine-2-carboxylic acid (Thz(2-COOH)), allylglycine (Agl), 4-cyano-2-aminobutyric acid (Cab), 2-pyridylalanine (2-Pal), 2-quinoylalanine (2-Qal), cyclobutylalanine (Cba), a phenylalanine analog, derivatives of lysine, ornithine (Orn) and α,γ-diaminobutyric acid (Dbu), stereoisomers thereof, and combinations thereof (see, e.g., Liu et al., Anal. Biochem., 295:9-16 (2001)). As such, the unnatural α-amino acids are present either as unnatural L-α-amino acids, unnatural D-α-amino acids, or combinations thereof.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally-occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins, 1993).

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody fragment) binds to a particular protein at least two times the background and does not substantially bind in a significant amount to other proteins present in the sample. Specific binding of an antibody fragment under such conditions may require an antibody fragment that is selected for its specificity for a particular protein or a protein but not its similar “sister” proteins. A variety of immunoassay formats may be used to select antibodies or fragments thereof that are specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

The term “therapeutically effective amount” refers to the amount of an antibody fragment or composition of the present invention that is capable of achieving a therapeutic effect in a subject in need thereof. For example, a therapeutically effective amount of an antibody fragment or composition of the present invention can be the amount that is capable of preventing or relieving one or more symptoms associated with a disease or disorder. One skilled in the art will appreciate that the antibody fragments and compositions of the present invention can be co-administered with other therapeutic agents such as anticancer, anti-inflammatory, immunosuppressive, antiviral, antibiotic, and/or antifungal agents.

The term “associates with each other,” when used in the context of two or more scFv molecules, refers to the pairing (e.g., interaction, non-covalent association, or specific binding) of the V_{H} domain of one scFv molecule with the V_{L} domain of another scFv molecule of a multivalent antibody fragment, such as a diabody, triabody, tetrabody, or minibody. For example, for a diabody, a first scFv molecule (containing a first V_{H} domain and a first V_{L} domain) forms a complex with a second scFv molecule (containing a second V_{H} domain and a second V_{L} domain) such that the first V_{H} domain non-covalently pairs with the second V_{L} domain and the second V_{H} domain non-covalently pairs with the first V_{L} domain.

As used herein, the term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of an antibody fragment or composition of the present invention for preventing or relieving one or more symptoms associated with a disease or disorder such as cancer or an inflammatory or autoimmune disease. By “co-administer” it is meant that an antibody fragment or composition of the present invention is administered at the same time, just prior to, or just after the administration of a second drug (e.g., anticancer agent, anti-inflammatory agent, immunosuppressive agent, antiviral agent, antibiotic, antifungal agent, etc.).

The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the present invention include, but are not limited to, fluorine 18 (¹⁸F), fluorine 19 (¹⁹F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁹⁵Rh) silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁹¹Tl), astatine 211 (²¹¹At), and bismuth 212 (²¹²Bi). As used herein, the “m” in ^{117m}Sn and ^{99m}Tc stands for the meta state. Additionally, naturally-occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶Ho, and ¹⁸⁸Re are examples of beta-emitting radionuclides. ⁶⁷Ga, ¹¹¹In, ^{99m}Tc, and ²⁹¹Tl are examples of gamma-emitting radionuclides. ⁵⁵Co, ⁶⁰Cu, ⁶¹CU, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

The term “subject” or “patient” typically refers to humans, but can also include other animals such as, e.g., other primates, rodents, canines, felines, equines, ovines, porcines, and the like.

### III. Detailed Description of the Embodiments

In some aspects, the present invention provides isolated multivalent antibody fragments that specifically bind αᵥβ₆ integrin comprising two or more scFv molecules that associate with each other, wherein each scFv molecule independently comprises the following structure:
(a) a light chain variable (V_{L}) region and a heavy chain variable (V_{H}) region of an antibody that specifically binds αᵥβ₆ integrin; and
(b) a peptide linker between the V_{L} region and the V_{H} region.

In particular embodiments, an imaging agent and/or a therapeutic agent is conjugated to the antibody fragments in a site-specific or nonsite-specific fashion.

In other aspects, the present invention provides compositions of and kits containing the isolated multivalent antibody fragments that specifically bind αᵥβ₆ integrin.

In yet other aspects, the present invention provides methods of in vivo imaging of a target tissue in a subject, the methods comprising: (a) administering to the subject in need of such imaging, an antibody fragment described herein or a composition thereof, wherein an imaging agent is conjugated to the antibody fragment; and (b) detecting the antibody fragment to determine where the antibody fragment is concentrated in the subject. In some embodiments, the imaging agent is a radionuclide.

In other aspects, the present invention provides methods of treating an αᵥβ₆ integrin-mediated disease or disorder in a subject in need thereof, the method comprising:

administering to the subject a therapeutically effective amount of an antibody fragment described herein or a composition thereof, wherein a therapeutic agent is conjugated to the antibody fragment. In some embodiments, the therapeutic agent is a radionuclide.

A. Antibody Fragments that Specifically Bind to αᵥβ₆ Integrin

The present invention provides multivalent antibody fragments, e.g., diabodies, tetrabodies, triabodies, and minibodies, that specifically bind to human αᵥβ₆ integrin. The antibody fragments described herein are advantageous over scFv fragments because they have longer blood circulation times and slower clearance rates.

The multivalent antibody fragments can be derived from the humanized intact anti-αᵥβ₆ integrin clone 6.3G9 (ATCC accession number PTA-3649). In some embodiments, the variable domains of the humanized 6.3G9 antibody are assembled to form the V_{H}-GGS-G_{L} module of the anti-αᵥβ₆ integrin antibody fragment. The nucleic acid sequences that encode the variable domains can be codon optimized. In some embodiments, the V_{H} chain of the multivalent antibody fragment has the amino acid sequence of SEQ ID NO:1. The peptide coding sequence of the V_{H} chain includes the DNA sequence set forth in SEQ ID NO:3. In some embodiments, the V_{L} chain of the multivalent antibody fragment has the amino acid sequence of SEQ ID NO:2. The corresponding peptide coding sequence of the V_{L} chain includes the DNA sequence set forth in SEQ ID NO:4. The anti-αᵥβ₆ integrin antibody fragments of the invention can also be functional variants of the V_{H} chain and V_{L} chain as defined above, including a heavy chain variable domain comprising an amino acid sequence of at least 90%, 91%, 92%, 93%, 94%, 95%, 96%, 97%, 98%, 99%, or 100% identity to SEQ ID NO: 1 and/or a light chain variable domain comprising an amino acid sequence of at least 90%, 91%, 92%, 93%, 94%, 95%, 96%, 97%, 98%, 99%, or 100% identity to SEQ ID NO:2. In certain instances, the peptides can comprise naturally-occurring amino acids and/or unnatural amino acids.

The V_{H} and V_{L} chains can be linked together by a peptide linker located between the V_{H} and V_{L} chains, thereby forming a scFv. In some embodiments, the length of the peptide linker between the V_{H} and V_{L} chains is shortened to less than 15 amino acids, e.g., 14, 13, 12, 11, 10, 9, 8, 7, 6, 5, 4, 3, 2, 1, or 0 amino acids, such that the natural association between the two chains is prevented.

In some embodiments, the isolated multivalent antibody fragment includes two or more scFv molecules that associate with each other. In some instances, the two or more scFv molecules are the same, e.g., the scFv molecule have the same V_{H} and V_{L} chains. In other instances, at least one of the two or more scFv molecules is different from the other molecules, e.g., at least two of the scFv molecules have different V_{H} and/or V_{L} chains. In some embodiments, each scFv molecule is appended with an additional cysteine or thiol residue at the C-terminus of each molecule.

In some embodiments, the V_{H} and V_{L} chains are forced to associate with separate scFv molecules to form bivalent dimers, termed diabodies. In some instances, a diabody contains a peptide linker of about 3 to about 15 amino acids in length, e.g., 3, 4, 5, 6, 7, 8, 9, 10, 11, 12, 13, 14 or 15 amino acids in length. In other instances, the peptide linker is about 3 to about 8 amino acids in length, e.g., 3, 4, 5, 6, 7 or 8, amino acids in length. In yet other instances, the peptide linker is about 3 to about 12 amino acids in length, e.g., 3, 4, 5, 6, 7, 8, 9, 10, 11 or 12 amino acids in length.

In some instances, each scFv molecule of the diabody has a cysteine or thiol residue inserted at the C-terminus of the scFv, thereby generating an anti-αᵥβ₆ integrin Cys-diabody (anti-αᵥβ₆ Cys-diabody).

The anti-αᵥβ₆ integrin diabodies can be larger in size (˜55 kDa) than scFvs. In some instances, the diabodies have longer blood circulation times and slower clearance compared to scFv molecules. The biological half-life of the diabody (between 2-6 hours) can be most compatible with radioisotopes with shorter half-lives such as [¹⁸F] (t_{1/2}=109.7 minutes, β⁺=98%).

In some embodiments, triabodies and tetrabodies can be formed by reducing the length of the flexible peptide linker that links the V_{H} and V_{L} chains. The transition between triabody and tetrabody depends on several factors, including orientation of the variable regions (V_{H}/V_{L} or V_{L}/V_{H}) as well as linker length, as both properties play important roles in how the two domains associate. Anti-αᵥβ₆ integrin triabodies can be formed by joining the variable domains with a peptide linker of about 1 to about 3 amino acids in length, e.g., 1, 2 or 3 amino acids in length. Anti-αᵥβ₆ integrin tetrabodies can be formed by joining the variable domains with a peptide linker of 0 to about 3 amino acids in length, e.g., 0, 1, 2 or 3 amino acids in length.

In some instances, each scFv molecule of the triabody or tetrabody has a cysteine or thiol residue inserted at the C-terminus of the scFv, thereby generating an anti-αᵥβ₆ integrin-cys triabody or tetrabody.

In some aspects, the trivalent and tetravalent binding triabodies and tetrabodies, respectively, increase their binding affinity for αᵥβ₆ integrin as well as their size (triabodies: ˜90 kDa, tetrabodies: ˜120 kDa) compared to other antibody fragments. This in turn can also improve their blood clearance rates.

In some embodiments, the two or more scFv fragments are fused to human antibody constant domains (C_{H}2 or C_{H}3) to form minibodies. The constant domains can be linked to the scFv fragments by a peptide linker of about 2 to about 12 amino acids in length, e.g., 2, 3, 4, 5, 6, 7, 8, 9, 10, 11, or 12 amino acids in length. These minibodies are generally larger in size (about 75 kDa) than a diabody and thus, have a longer blood circulation time. Minibodies may have the same bivalent binding affinity as their corresponding diabody.

Methods for generating the multivalent antibody fragments described herein utilize routine techniques in the field of molecular biology. For instance, nucleic acids having, for example, the sequences of SEQ ID NOS. 3 and 4 can be generated by PCR, restriction enzyme digestion, molecular cloning, site-directed mutagenesis, etc. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); D. M. Glover et al. ed., “DNA Cloning”, 2nd ed., Vol. 1, (The Practical Approach Series), IRL Press, Oxford University Press (1995) and M. A. Innis et al. ed., “PCR Protocols”, Academic Press, New York (1990) (PCR); and Current Protocols in Molecular Biology (Ausubel et al., eds., 1994)). Commercially available agents and kits can be used in accordance with method described herein. In some embodiments, the antibody fragment is made recombinantly in a host cell.

The antigen-binding V_{L} (variable light chain) and V_{H} (variable heavy chain) sequences for the αᵥβ₆ integrin antibody may be obtained by a variety of molecular cloning procedures, such as RT-PCR, 5′-RACE, and cDNA library screening. The V genes can be cloned by PCR amplification and sequenced. To confirm their authenticity, the cloned V_{L} and V_{H} genes can be expressed in cell culture as a chimeric antibody as described by Orlandi et al., (Proc. Natl. Acad. Sci., USA, 86: 3833 (1989)). In some instances, the cloned V_{L} and V_{H} genes can be subcloned into an expression vector and the resulting expression construct can be transfected into a host cell.

In some embodiments, the antibody fragments are generated in a cell-based protein synthesis system or a cell-free protein synthesis system. Useful cell-based protein synthesis systems include those utilizing bacteria cells (e.g., Escherichia coli), mammalian cells (e.g., CHO cells, HEK293 cells, myeloma cells), human cells; insect cells, or plant cells as host cells.

The antibody fragment can be obtained by the expression in a host cell. In some instances, the antibody fragment is secreted and collected from the culture medium. In other instances, the fragment may be collected from cell lysate.

Purification of the antibody fragment may be carried out by any method known to those skilled in the art such as centrifugation, hydroxyapatite chromatography, gel electrophoresis, dialysis, separation by ion-exchange chromatography, ethanol precipitation, reverse phase HPLC, silica chromatography, heparin-sepharose chromatography, anion- or cation-resin chromatography such as polyaspartic acid column, chromato-focusing, SDS-PAGE, precipitation with ammonium sulfate, and affinity chromatography. The affinity chromatography, which utilizes affinity with a peptide tag (e.g., His tag) of the antibody fragment, is one of the preferred purification techniques with a high efficiency.

B. Incorporating Unnatural Amino Acids into Multivalent Antibody Fragments

In some embodiments, the multivalent antibody fragments contain one or more, e.g., 1, 2, 3, 4, 5, 6, 7, 8, 9, 10 or more unnatural amino acids. In some instances, the location of the introduced unnatural amino acid is distant from the CDR regions or other regions that are necessary for antigen specificity or selectivity. In particular instances, the unnatural amino acid is located at the C-terminal of the scFv molecules of the antibody fragment. In some embodiments, the unnatural amino acid is added to replace a corresponding natural amino acid. (e.g., a lysine derivative is introduced to replace a lysine residue). The unnatural amino acid may be added at a location (position) such that it is least likely to interfere with antigen recognition. Additionally, the unnatural amino acid is placed to reduce any deleterious effects caused by its addition to the multivalent antibody fragment. Methods for generating antibodies containing unnatural amino acids are described below and in, for example, Axup et al., Proc Natl Acad Sci USA, 109(40):16101-16106, 2012; Kim et al., Curr Opin Chem Biol, 17(3):412-419, 2013; and Tian et al., Proc Natl Acad Sci USA, 111(5):1766-1771, 2014.

To incorporate an unnatural amino acid into a nascent polypeptide, a selector codon can be included in the coding sequence of the polypeptide. In addition, the selected unnatural amino acid should be metabolically stable. It also cannot be the substrate for any endogenous aminoacyl-tRNA synthetases (RSs). Also, an orthogonal aminoacyl-tRNA synthetases as well as an orthogonal tRNA which recognize the new unnatural amino acid should be used.

Detailed descriptions of exemplary unnatural amino acids and the incorporation of such unnatural amino acids into polypeptide are found in, for example, U.S. Pat. Nos. 7,083,970; 7,354,761; 7,368,275; 7,638,300; 7,713,721; 7,915,025; 8,012,739; 8,030,074; 8,114,648; 8,173,364; 8,173,392; and 8,183,012, the disclosures of which are herein incorporated by reference in their entirety for all purposes.

The unnatural amino acids of the present invention encompass a variety of substances. For example, they include, but are not limited to, such molecules as: an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine. Additionally, other examples include, but are not limited to, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged amino acid; a photoisomerizable amino acid; a biotin or biotin-analogue containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an .alpha.-hydroxy containing acid; an amino thio acid containing amino acid; an α,α-disubstituted amino acid; a β-amino acid; and a cyclic amino acid other than proline.

Selector codons expand the genetic codon framework of protein biosynthetic machinery. For example, a selector codon includes, e.g., a unique three base codon (composed of natural or unnatural bases), a nonsense codon (such as a stop codon, e.g., an amber codon, or an opal codon), an unnatural codon, a rare codon, a codon comprising at least four bases, a codon comprising at least five bases, a codon comprising at least six bases, or the like. In some embodiments, a selector codon is introduced into a nucleic acid that encodes the anti-antigen-binding V_{L} and/or V_{H} chain for the anti-αᵥβ₆ integrin antibody fragment. In some instances, a selector codon replaces a codon that encodes a natural amino acid.

In some embodiments, the antibody fragment is synthesized in the presence of an orthogonal aminoacyl-tRNA synthetase/tRNA pair (O-RS/O-tRNA pair) that recognizes the selector codon and the unnatural amino acid. The O-tRNA and the O-RS can be derived by mutation of a naturally occurring tRNA and RS from a variety of organisms. For instance, the O-tRNA and O-RS can be derived from at least one organism, where the organism is a prokaryotic organism, e.g., Methanococcus jannaschii, Methanobacterium thermoautotrophicum, Halobacteriumi, Escherichia coli, A. fulgidus, P. furiosus, P. horikoshii, A. pernix, T. thermophilus, or the like. Optionally, the organism can be a eukaryotic organism, e.g., plants (e.g., complex plants such as monocots, or dicots), algea, fungi (e.g., yeast, etc.), animals (e.g., mammals, insects, arthropods, etc.), insects, protists, or the like. Alternatively, the O-tRNA can be derived by mutation of a naturally occurring tRNA from a first organism and the O-RS is derived by mutation of a naturally occurring RS from a second organism. In other instances, the O-tRNA and O-RS can be derived from a mutated tRNA and mutated RS. Methods for producing at least one recombinant orthogonal aminoacyl-tRNA synthetase (O-RS) pairs are described in detail in, for example, U.S. Pat. Nos. 7,083,970; 7,354,761; 7,713,721; and 8,183,012, the disclosures of which are herein incorporated by reference in their entirety for all purposes.

In some embodiments, the host cell used to recombinantly produce the anti-αᵥβ₆ integrin antibody fragment of the present invention comprises the nucleic acid that includes a selector codon and that encodes the antibody fragment, an orthogonal tRNA that functions in the cell and recognizes the selector codon, and an orthogonal aminoacyl tRNA synthetase that preferentially aminoacylates the orthogonal tRNA with the selected unnatural amino acid. In some instances, the unnatural amino acid is found in the culture medium used for the growth of the host cell.

C. Methods for Labeling Multivalent Antibody Fragments with Radionuclides

The anti-αᵥβ₆ antibody fragments described herein can be labeled with a radionuclide by direct conjugation or indirect conjugation. The radionuclide can be conjugated at a site-specific amino acid residue (e.g., a natural amino acid or an unnatural amino acid) of the antibody fragment. Optionally, the radionuclide can be conjugated nonsite-specifically. In some embodiments, the radionuclide is conjugated to the cysteine or thiol residue at the C-terminus of the anti-αᵥβ₆ antibody fragment. In some embodiments, the radionuclide is conjugated to one or more unnatural amino acids of the anti-αᵥβ₆ antibody fragment or anti-αᵥβ₆ Cys-antibody fragment.

Radionuclides differ based on their characteristics, which include half-life, energy emission characteristics, and type of decay. Thus radionuclides may be selected based on desired characteristics suitable for use in in vivo imaging and/or for therapeutic applications. For example, gamma emitters are generally used diagnostically and beta emitters are generally used therapeutically. However, some radionuclides are both gamma emitters and beta emitters, and thus, may be suitable for both uses by altering the amount of radioactivity used (the total and/or specific activity). Radionuclides that are useful for the present invention include, but are not limited to ¹¹C, ¹³N, ¹⁵O, ¹⁸F, ¹⁹F, ³²P, ³³P, ⁴⁷C, ⁵¹Cr, ⁵⁷Co, ⁵⁸Co, ⁵⁹Fe, ⁶¹Cu, ⁶²Cu, ⁶⁴Cu, ⁶⁷Cu, ⁶⁷Ga, ⁶⁸Ga, ⁷⁵Br, ⁷⁵Se, ⁷⁵Se, ⁷⁶Br ⁷⁷As, ⁷⁷Br, ^{80m}Br, ⁸⁹Sr, ⁹⁰Y, ⁹⁵Ru, ⁹⁷Ru, ⁹⁹Mo, ^{99m}Tc, ^{103m}Rh, ¹⁰³Ru, ¹⁰⁵Ru, ¹⁰⁷Hg, ¹⁰⁹Pd, ¹⁰⁹Pt, ¹¹¹Ag, ¹¹¹In, ¹¹⁹Sb, ^{121m}Te, ^{122m}Te, ¹²⁴I, ¹²⁵I, ^{125m}Te, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Pb, ²¹⁷At, ²¹⁹Rn, ²²¹Fr, ²²³Ra, ²²⁴Ac, ²²⁵Ac, ²²⁷Th and ²⁵⁵Fm. These radionuclides are well known in the art, as are methods of making them and labeling proteins (e.g., antibodies and fragments thereof) with them. The selected radionuclide can be introduced into the antibody fragment of the present invention at one or more sites, e.g., 1, 2, 3, 4, 5, 6, 7, 8, 9, 10 or more sites.

There are numerous methods for labeling antibody fragments (or more generally, proteins) with radionuclides described in the art and the particular method used depends on the antibody fragment, the radionuclide and the application of the resulting radiolabeled protein. In some embodiments, the selected radionuclide is incorporated into the anti-αᵥβ₆ antibody fragment during protein synthesis, e.g., chemical synthesis or recombinant synthesis. In other embodiments, the radionuclide is added via a reactive-thiol of a cysteine in the protein. For example, the radionuclide can be added to the exposed cysteine residue at the C-terminus of the anti-αᵥβ₆ Cys antibody fragment. In some embodiments, an amino acid substitution, such as a substitution of an unnatural amino acid for a natural amino acid, is introduced into the anti-αᵥβ₆ antibody fragment to provide a site for labeling.

In some embodiments, suitable methods for radiolabeling anti-αᵥβ₆ antibody fragments include using a crosslinking agent such as, but not limited to, succinimidyl 4-(N-maleimidomethyl) cyclohexane-1-carboxylate (SMCC), m-maleimidobenzoyl-N-hydroxysuccinimide ester (MBS), N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC), 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate]hexanoate (LC-SPDP), disuccinimidyl suberate (DSS), bismaleimidohexane (BMH), dimethylpimelimidate-2 HCl (DMP), bis-[B-(4-azidosalicylamido)ethyl]disulfide (BASED), N-succinimidyl-6(4′-azido-2′-nitrophenylamino)hexanoate (SANPAH), N-hydroxysuccinimide (NHS) or its water soluble analog N-hydroxysulfosuccinimide (sulfo-NHS), and other crosslinkers that contain a primary amine reactive group.

Alternatively, the radionuclide (e.g., therapeutic agent and/or imaging agent) can be attached to the antibody fragment using click chemistry reactions. Various forms of click chemistry reactions are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions, carbonyl chemistry formation of urea compounds and reactions involving carbon-carbon double bonds, such as alkynes in thiol-yne reactions.

In some embodiments, the radionuclide, e.g., ¹⁸F, ¹⁹F, ¹²⁴I, ¹²⁵I, ¹³¹I, and mixtures thereof, is attached to the anti-αᵥβ₆ antibody fragment by direct conjugation. For instance, ¹²⁴I can be directly radiolabeled to the antibody fragment. ¹²⁴I can be added to tyrosyl residues using solid-phase oxidation.

In other embodiments, the radionuclide is attached to the anti-αᵥβ₆ antibody fragment by indirect conjugation. Radiometals such as ⁶⁴Cu and ⁸⁹Zr can be indirectly conjugated through the use of a bifunctional chelating agent. The bifunctional chelate can form multiple coordinate bonds with the radiometal, acts like a small cage to “trap” the radiometal within the complex. The bifunctional chelate can feature a chemical function group (activated ester, maleimide, etc), which can be conjugated to the antibody fragment via lysine side-chains, or cysteine thiol groups. In some embodiments, multiple chelations are obtained globally throughout the antibody fragment, depending on the number of lysine or cysteine residues that are solvent exposed. In some instances, ⁶⁴Cu is conjugated to the antibody fragments using the bifunctional chelate 1, 4, 7, 10-tetraazacyclododecane-N,N′,N″,N′″-tetraacetic acid (DOTA). In other embodiments, [⁸⁹Zr] is conjugated to the antibody fragments using desferrioxamine (DFO). Other suitable chelating agents include, but are not limited to, DOTA, NOTA, NOTA-TCO, BAD, TETA, DTPA, EDTA, NTA, HDTA, their phosphonate analogs, and mixtures thereof

Radiometals such as ¹⁸F can be indirectly conjugated through the use of a prosthetic group. In some embodiments, ¹⁸F is incorporated into the prosthetic group, followed by attachment to the antibody fragment. The prosthetic group is selected such that it has minimal effects on the binding and specificity to the antibody fragment towards its target antigen. In some instances, the prosthetic group for radiofluorination is N-succinimidyl-4-[18F] fluorobenzoate ([¹⁸F] SFB). [¹⁸F] SFB can be synthesized from the production of [¹⁸F] with a biomedical cyclotron and through an 4-[¹⁸F]fluorobenzoic acid ([¹⁸F]FBA) intermediate. The [¹⁸F]FBA can then be converted to the activated ester using N,N,N′,N′-Tetramethyl-O—(N-succinimidyl)uronium tetrafluoroborate (TSTU). In some embodiments, the antibody fragment is radiolabeled with [¹⁸F]SFB on exposed lysine residues.

A radiolabel can be added to an antibody fragment by firstly introducing cysteine residues into expressed regions of the variable domain framework to promote or induce the formation of disulfide bond. In some embodiments, anti-αᵥβ₆ integrin antibody fragment (e.g., diabody) contains at least one additional cysteine or thiol residue at the C-terminus of the fragment. The bifunctional chelator 1,4,7-triazacyclonane-1,4,7-triacetic acid (NOTA) can be used to conjugate ¹⁸F, ⁶⁴Cu, ⁶⁸Ga, or ¹¹¹In to the anti-αᵥβ₆ Cys antibody fragment.

In some embodiments, a radiolabel is site-specifically conjugated to an unnatural amino acid of the antibody fragment. For instance, click chemistry methods such as TTCO ligation can be used to conjugate a radionuclide to the UAA of the antibody fragment.

D. Methods for In Vivo Imaging Target Tissue in a Subject Using Multivalent Antibody Fragments Labeled with Imaging Agents

In another aspect of the present invention, the anti-αᵥβ₆ antibody fragment described herein can be used for in vivo imaging of diseased cells or tissue in various biomedical applications including, but not limited to, imaging of tumors, tomographic imaging of organs, monitoring of organ functions, coronary angiography, fluorescence endoscopy, laser guided surgery, photoacoustic and sonofluorescence methods, and the like. In some embodiments, the antibody fragments of the invention are useful for the detection of the presence of tumors and other abnormalities by monitoring where a particular conjugate is concentrated in a subject. In some instances, the anti-αᵥβ₆ antibody fragment is conjugated to an imaging agent.

In some embodiments, the imaging agent is selected from the group consisting of a radionuclide, biotin, a fluorophore, a fluorescent protein, an antibody, horseradish peroxidase, alkaline phosphatase, and combinations thereof. In certain embodiments, the radionuclide is selected from the group consisting of ¹¹C, ¹³N, ¹⁵O, ¹⁸F, ¹⁹F, ⁶¹Cu, ⁶²Cu, ⁶⁴Cu, ⁶⁷Cu, ⁶⁸Ga, ¹¹¹In, ¹²⁴I, ¹²⁵I, and ¹³¹I. In certain instances, the radionuclide is attached via a prosthetic group or a bifunctional chelator to the anti-αᵥβ₆ antibody fragment. Non-limiting examples of prosthetic groups or bifunctional chelators include benzoyl groups (e.g., fluorobenzoic acid (FBA)), fluoropropionic acid (FPA), pyridine (Py), dipyridyl-tetrazine (Tz), trans-cyclooctene (TCO), derivatives thereof, and combinations thereof. For example, 4-[¹⁸F]-fluorobenzoic acid ([¹⁸F]FBA) or 4-[¹⁹F]-fluorobenzoic acid ([¹⁹F]FBA) can be used to radiolabel the anti-αvβ6 antibody fragment of the present invention. Additional non-limiting examples of chelating agents include macrocyclic metal chelators such as DOTA (1,4,7,10-tetraazacyclododecane-N,N′,N″,N′″-tetraacetic acid), NOTA (1,4,7-triazacyclononane-N,N′,N″-triacetic acid), DTPA (diethylenetriaminepentaacetic anhydride), TETA (1,4,8,11-tetraazacyclotetradecane-N,N′,N″,N′″-tetraacetic acid), and DTTA (N-(p-isothiocyanatobenzyl)-diethylenetriamine-N,N′,N″,N′″-tetraacetic acid).

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol., 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, mOrange, mBanana, mHoneydew, and mTangerine. Additional DsRed variants are described in, e.g., Wang et al., Proc. Natl. Acad. Sci. USA., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett, 580:2495-2502 (2006).

In other embodiments, the imaging agent that is bound to an antibody fragment of the present invention comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

In instances where a radionuclide comprises the imaging agent, detection occurs when radiation from the radionuclide is used to determine where the anti-αᵥβ₆ antibody fragment is concentrated in the subject. In instances where a fluorophore or fluorescent protein comprises the imaging agent, detection occurs when fluorescence from the fluorophore or fluorescent protein is used to determine where the anti-αᵥβ₆ antibody fragment is concentrated in the subject.

In other embodiments, the anti-αᵥβ₆ antibody fragment is detected by Magnetic Resonance Imaging (MRI), Magnetic Resonance Spectroscopy (MRS), Single Photon Emission Computerized Tomography (SPECT), Positron Emission Tomography (PET), or optical imaging. In yet other embodiments, the conjugate is detected for the diagnosis or prognosis of a disease or disorder mediated by the integrin. In certain embodiments, the disease or disorder is associated with the expression, overexpression, and/or activation of the integrin. In preferred embodiments, the disease or disorder is an αᵥβ₆ integrin-mediated disease or disorder, e.g., the antibody fragment is detected for the diagnosis or prognosis of an αᵥβ₆ integrin-mediated disease or disorder.

In an additional aspect, the present invention provides a method for imaging epithelial cells expressing or overexpressing αᵥβ₆integrin in the body of a subject, the method comprising administering to the subject a therapeutically effective amount of the anti-αᵥβ₆ antibody fragment or composition as described herein. The method is particularly useful for the imaging of chronic fibrosis, chronic obstructive pulmonary disease (COPD), lung emphysema, chronic wounding skin disease (e.g., epidermolysis bullosa), or epithelial tumor cells. For example, the method of imaging αᵥβ₆-overexpressing epithelial cells may include linking the anti-αᵥβ₆ antibody fragment to a fluorescent or radionuclide probe, and incorporating the resulting antibody fragment into a suitable dosage form such that upon administration the αᵥβ₆ integrin-binding antibody fragment may be visualized by its fluorescent tag or radioactive tag, respectively.

The anti-αᵥβ₆ integrin antibody fragment can be administered either systemically or locally to the tumor, organ, or tissue to be imaged, prior to the imaging procedure. Generally, the antibody fragments are administered in doses effective to achieve the desired optical image of a tumor, tissue, or organ. Such doses may vary widely, depending upon the antibody fragment employed, the tumor, tissue, or organ subjected to the imaging procedure, the imaging equipment being used, and the like.

A detectable response generally refers to a change in, or occurrence of, an optical signal that is detectable either by observation or instrumentally. In certain instances, the detectable response is radioactivity (i.e., radiation), including alpha particles, beta particles, nucleons, electrons, positrons, neutrinos, and gamma rays emitted by a radioactive substance such as a radionuclide. In certain other instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ).

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled conjugate of the present invention. Positron emission tomography (PET) is another suitable technique for detecting radiation in a subject. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the antibody fragment is concentrated in a subject, with such concentration being an indicator of the location of a tumor or tumor cells.

Non-invasive fluorescence imaging of animals and humans can also provide in vivo diagnostic or prognostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al., IEEE Transactions on Biomedical Engineering, 48:1034-1041 (2001)), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, and signal amplification using photomultiplier tubes.

E. Methods for Treating αᵥβ₆ Integrin-Mediated Diseases or Disorders Using Multivalent Antibody Fragments Labeled with Therapeutic Agents

In a related aspect, the present invention provides a method for treating an αᵥβ₆ integrin-mediated disease or disorder in a subject in need thereof, the method comprising administering to the subject a therapeutically effective amount of any of the anti-αᵥβ₆ antibody fragments described herein or a composition thereof, wherein a therapeutic agent is conjugated to the antibody fragment.

In certain embodiments, the disease or disorder is associated with the expression, overexpression, and/or activation of αᵥβ₆ integrin. Non-limiting examples of αᵥβ₆ integrin-mediated diseases or disorders include cancer, inflammatory diseases, autoimmune diseases, chronic fibrosis, chronic obstructive pulmonary disease (COPD), lung emphysema, and chronic wounding skin disease. In some instances, the αᵥβ₆ integrin-mediated disease or disorder is ovarian cancer, breast cancer, lung cancer, bladder cancer, thyroid cancer, liver cancer, pleural cancer, pancreatic cancer, cervical cancer, prostate cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, skin cancer, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma. In other embodiments, a therapeutically effective amount of an anti-αᵥβ₆ integrin antibody fragment or composition thereof is an amount sufficient for achieving a therapeutic benefit in the subject. In yet other embodiments, a therapeutically effective amount of the antibody fragment or composition thereof is an amount sufficient to target delivery of the therapeutic agent to a cell expressing the integrin.

In some embodiments, the therapeutic agent is selected from the group consisting of a radionuclide, a pro-apoptotic peptide, a nanoparticle, a chemotherapeutic agent, a nanodroplet, a liposomal drug, a cytokine, and combinations thereof. The anti-αᵥβ₆ antibody fragment can be labeled with a therapeutic agent (e.g., radionuclide) using any available method and chemistry described above. Association or conjugation of the agent may be directly or via a coupling agent (e.g., chelating agent) or linker.

In certain embodiments, the therapeutic agent is a radionuclide selected from the group consisting of ¹³¹I, ³⁷Cu, ²¹¹At, ²¹²Bi, ²¹³Bi, ²²⁵Ac, ⁹⁰Y and ¹⁷⁷Lu. In certain instances, the radionuclide is attached via a chelating agent to the anti-αᵥβ₆ antibody fragment. Non-limiting examples of chelating agents include macrocyclic metal chelators such as DOTA (1,4,7,10-tetraazacyclododecane-N,N′,N″,N′″-tetraacetic acid), NOTA (1,4,7-triazacyclononane-N,N′,N″-triacetic acid), DTPA (diethylenetriaminepentaacetic anhydride), TETA (1,4,8,11-tetraazacyclotetradecane-N,N′,N″,N′″-tetraacetic acid), and DTTA (N-(p-isothiocyanatobenzyl)-diethylenetriamine-N,N′,N″,N′″-tetraacetic acid).

In some embodiments, the therapeutic agent comprises a radionuclide that is damaging or cytotoxic to cells and the anti-αᵥβ₆ antibody fragment targets the therapeutic agent preferentially to cancerous cells. In certain embodiments, the radionuclide is an alpha or beta emitting radionuclide. In certain embodiments, the radionuclide is selected from the group consisting of ¹³¹I, ³⁷Cu, ²¹¹At, ²¹²Bi, ²¹³Bi, ²²⁵Ac, ⁹⁰Y and ¹⁷⁷Lu. In certain embodiments, the radionuclide is ⁹⁰Y.

Administration of the anti-αᵥβ₆ antibody fragment of the present invention with a suitable pharmaceutical excipient as necessary can be carried out via any of the accepted modes of administration. Thus, administration can be, for example, intravenous, topical, subcutaneous, transcutaneous, transdermal, intramuscular, oral, intra joint, parenteral, intra-arteriole, intradermal, intraventricular, intracranial, intraperitoneal, intralesional, intranasal, rectal, vaginal, or by inhalation. Moreover, where injection is to treat a tumor, administration may be directly to the tumor and/or into tissues surrounding the tumor.

The compositions containing the anti-αᵥβ₆ antibody fragment or a combination of anti-αᵥβ₆ antibody fragments of the present invention may be administered repeatedly, e.g., at least 2, 3, 4, 5, 6, 7, 8, or more times, or the composition may be administered by continuous infusion. Suitable sites of administration include, but are not limited to, dermal, mucosal, bronchial, gastrointestinal, anal, vaginal, eye, and ear. The formulations may take the form of solid, semi-solid, lyophilized powder, or liquid dosage forms, such as, for example, tablets, pills, lozenges, capsules, powders, solutions, suspensions, emulsions, suppositories, retention enemas, creams, ointments, lotions, gels, aerosols, or the like, preferably in unit dosage forms suitable for simple administration of precise dosages.

The term “unit dosage form” refers to physically discrete units suitable as unitary dosages for human subjects and other mammals (e.g., dogs), each unit containing a predetermined quantity of active material calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of an anti-αᵥβ₆ antibody fragment or a combination of anti-αᵥβ₆ antibody fragments.

Methods for preparing such dosage forms are known to those skilled in the art (see, for example, REMINGTON'S PHARMACEUTICAL SCIENCES, 18TH ED., Mack Publishing Co., Easton, Pa. (1990)). The composition to be administered contains a quantity of the anti-αᵥβ₆ antibody fragment or combination of anti-αᵥβ₆ antibody fragments in a pharmaceutically effective amount for imaging a tumor, organ, or tissue or for relief of a condition being treated, when administered in accordance with the teachings of this invention. In addition, pharmaceutically acceptable salts of the antibody fragments of the present invention (e.g., acid addition salts) may be prepared and included in the compositions using standard procedures known to those skilled in the art of synthetic organic chemistry and described, e.g., by March, Advanced Organic Chemistry: Reactions, Mechanisms and Structure, 4th Ed., New York, Wiley-Interscience (1992).

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, about 0.1% to about 75%, about 0.1% to 50%, or about 0.1% to 10% by weight of an antibody fragment of the present invention or a combination thereof, with the remainder consisting of suitable pharmaceutical carrier and/or excipients. Appropriate excipients can be tailored to the particular composition and route of administration by methods well known in the art. See, e.g., REMINGTON'S PHARMACEUTICAL SCIENCES, supra.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The compositions can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; coloring agents; and flavoring agents. The compositions may also comprise biodegradable polymer beads, dextran, and cyclodextrin inclusion complexes.

For oral administration, the compositions can be in the form of tablets, lozenges, capsules, emulsions, suspensions, solutions, syrups, sprays, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the pharmaceutical compositions take the form of a pill, tablet, or capsule, and thus, the composition can contain, along with the antibody fragment or combination of antibody fragments, any of the following: a diluent such as lactose, sucrose, dicalcium phosphate, and the like; a disintegrant such as starch or derivatives thereof a lubricant such as magnesium stearate and the like; and a binder such a starch, gum acacia, polyvinylpyrrolidone, gelatin, cellulose and derivatives thereof. The antibody fragments can also be formulated into a suppository disposed, for example, in a polyethylene glycol (PEG) carrier.

Liquid compositions can be prepared by dissolving or dispersing an antibody fragment or a combination of antibody fragments and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. The antibody fragments of the present invention can also be formulated into a retention enema.

For topical administration, the compositions of the present invention can be in the form of emulsions, lotions, gels, creams, jellies, solutions, suspensions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Generally, administered dosages will be effective to deliver picomolar to micromolar concentrations of the antibody fragment to the appropriate site or sites. However, one of ordinary skill in the art understands that the dose administered will vary depending on a number of factors, including, but not limited to, the particular antibody fragment or set of antibody fragments to be administered, the mode of administration, the type of application (e.g., imaging, diagnostic, prognostic, therapeutic, etc.), the age of the patient, and the physical condition of the patient. Preferably, the smallest dose and concentration required to produce the desired result should be used. Dosage should be appropriately adjusted for children, the elderly, debilitated patients, and patients with cardiac and/or liver disease. Further guidance can be obtained from studies known in the art using experimental animal models for evaluating dosage. However, the increased metabolic stability, tumor retention, and tumor to blood ratios associated with the antibody fragments of the present invention permits a wider margin of safety for dosage concentrations and for repeated dosing.

One skilled in the art will also appreciate that the antibody fragments of the present invention can be co-administered with other therapeutic agents for the treatment of cancer. Suitable anti-cancer agents for combination therapy include, without limitation, cytotoxins and agents such as antimetabolites, alkylating agents, anthracyclines, antibiotics, antimitotic agents, procarbazine, hydroxyurea, asparaginase, corticosteroids, interferons, radiopharmaceuticals, peptides with anti-tumor activity such as TNF-α, pharmaceutically acceptable salts thereof derivatives thereof, prodrugs thereof, and combinations thereof. For example, a pharmaceutical composition comprising one or more antibody fragments of the present invention may be administered to a patient before, during, or after administration of an anti-cancer agent or combination of anti-cancer agents either before, during, or after chemotherapy. Treatment with the antibody fragment after chemotherapy may be particularly useful for reducing and/or preventing recurrence of the tumor or metastasis.

### IV. Examples

The following examples are offered to illustrate, but not to limit the claimed invention.

### Example 1

### Methods for Generating Multivalent Antibody Fragments that Specifically Bind αvβ6 Integrin

This example illustrates the design and construction of two novel humanized diabodies specific to αᵥβ₆ integrin, the anti-αᵥβ₆ diabody and a disulfide-stabilized anti-αᵥβ₆ Cys-diabody. Each is derived from the humanized intact anti-αvβ6 antibody clone 6.3G9.77. The variable domains of the 6.3G9 antibody were assembled in the format of VH-G2S-VL to generate the anti-αᵥβ₆ diabody. The construction of the engineered anti-αᵥβ₆ Cys-diabody was based on the format of the parental anti-αᵥβ₆ diabody, with the addition of engineered cysteine residues appended onto the C-termini. Each of these novel antibody fragments was expressed from stable mammalian cell lines and biochemical characterization assays were conducted on each antibody fragment to confirm molecular weight, purity, and identity.

The disulfide-stabilized Cys-diabody provides two unique advantages over the parental non-covalently bound diabody. Firstly, the disulfide bridge serves to covalently bind the two single chain FAT (scFv) fragments together to promote improved stability and retention of bivalent antigen binding. Secondly, the engineered cysteine residues provide a location which, upon reduction of the disulfide bridge, can enable site-specific modification of the Cys-diabody in a region distant from the antigen binding sites.

### Materials and Methods

A. Cell Lines and Cell Culture Reagents

The 293-F cell line (Invitrogen) was maintained in non-selective growth media consisting of Dulbecco's Modified Eagle Medium (DMEM, Mediatech) supplemented with 10% fetal bovine serum (FBS, Invitrogen), 1% non-essential amino acids (NEAA, Invitrogen), and 1% penicillin-streptomycin-L-glutamine (PSG, Invitrogen). Stable cell lines expressing anti-αᵥβ₆ diabody were maintained in selective growth media comprised of DMEM, 10% FBS, 1% NEAA, 1% PSG, and 300 μg/mL Zeocin (Invitrogen). Production media consisted of Opti-MEM (Invitrogen) supplemented with 1% NEAA, 1% PSG, 100 μg/mL Zeocin. The Dx3Puro and Dx3Puroβ₆ cell lines were cultured in DMEM supplemented with 10% FBS and 1% PSG. The Dx3Puroβ₆ cell line was generated by retroviral transfection with human β₆ cDNA as previously described. All cell lines were cultured in Cellstar™ cell culture T-flasks (Greiner Bio-One) at 37° C. in a humidified incubator with 5% CO₂ unless otherwise noted. All chemicals were purchased from Sigma-Aldrich (St. Louis, Mo.), Acros (Geel, Belgium) or Fluka (St. Louis, Mo.).

B. Design and Construction of Anti-αᵥβ₆ Diabody and Anti-αᵥβ₆ Cys-Diabody

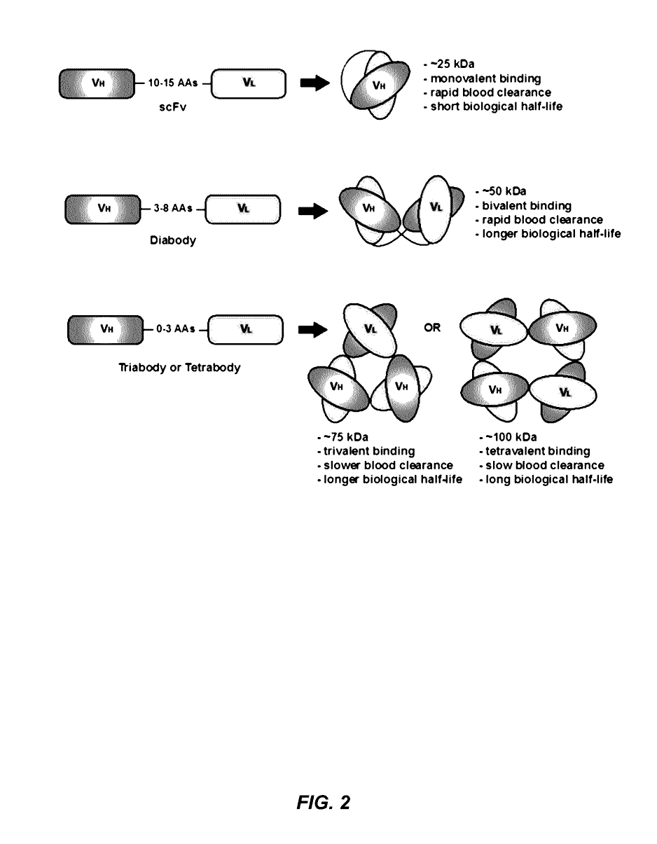

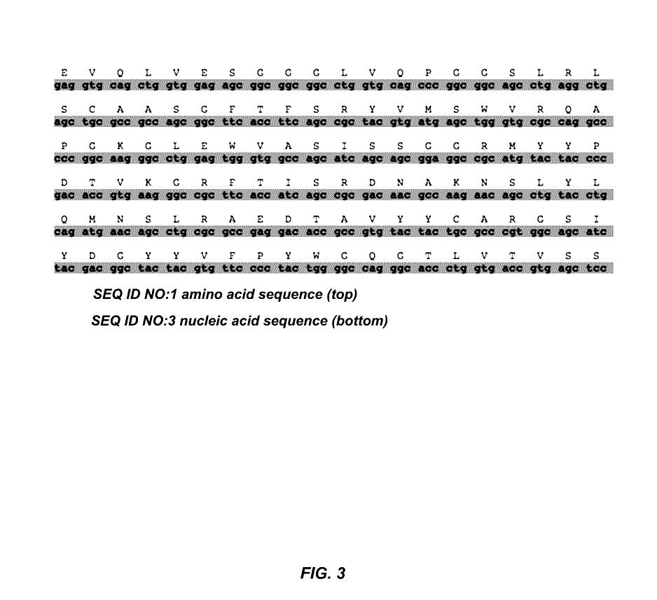

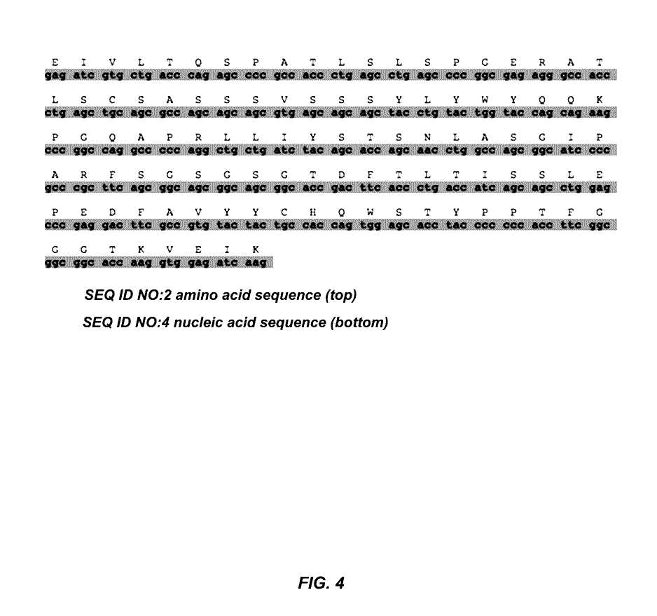

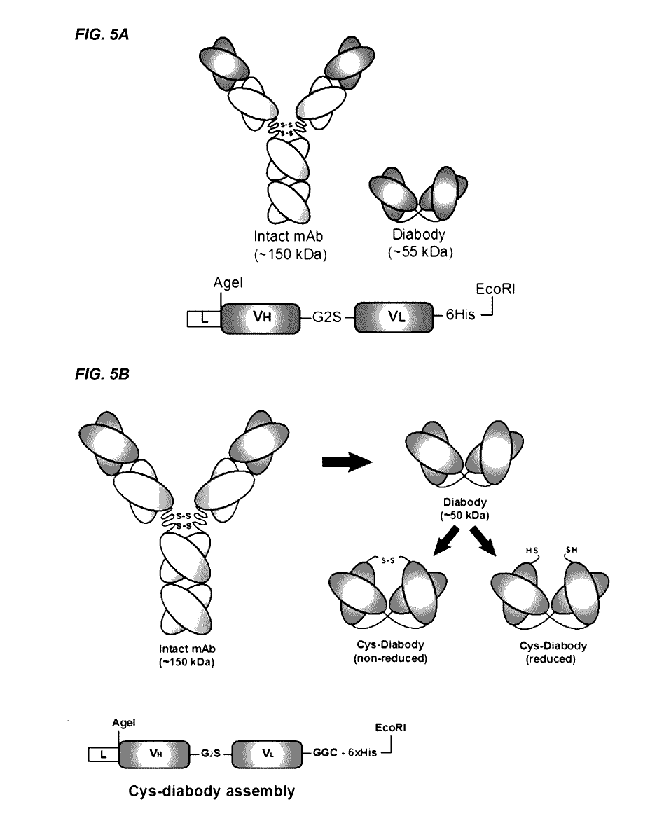

For the anti-αᵥβ₆ diabody, the DNA sequences of the variable heavy (V_{H}) and variable light (V_{L}) chains were derived from the intact humanized hu6.3G9 antibody (FIGS. 3 and 4). DNA was mammalian optimized and assembled to encode the V_{H} and V_{L} domains, separated by a flexible three amino acid linker (G2S), and appended with a 6×-His tag at the C-terminus (FIGS. 2 and 5A). Polymerase chain reaction (PCR) was used to incorporate the AgeI and EcoRI restriction sites at the N-terminus and C-terminus, respectively and the DNA was purified on a 1% agarose gel. The expression plasmid pSecTag-2A (Invitrogen) was mutated to include the AgeI restriction site at the C-terminus of the leader sequence using the Quick Change Site-Directed Mutagenesis kit (Agilent) to create pSecTag-2A-AgeI. The anti-αᵥβ₆ diabody construct was subcloned into the pSecTag-2A-AgeI plasmid between AgeI and EcoRI using standard molecular cloning techniques to create pSecTag-B6DiaR-G2S. Subcloning was verified by restriction digestion.

For the anti-αᵥβ₆ Cys-diabody, the original template DNA from the pSecTag-B6DiaR-G2S plasmid was used to generate the anti-αᵥβ₆ Cys-diabody. Using mutagenic polymerase chain reaction (PCR) primers and the site-directed mutagenesis kit (Stratagene), a cysteine residue separated by glycine spacers were appended to the C-terminus of the original anti-αᵥβ₆ diabody (FIG. 5B). The remaining original features of the anti-αᵥβ₆ diabody were left unchanged. DNA sequencing was used to verify insertion of the GGC residues, creating plasmid pSecTag-B6DiaR-G2S-Cys. Plasmid DNA was purified using a plasmid plus midi kit (Qiagen).

C. Generation of Stable Cell Lines Expressing Anti-αᵥβ₆ Diabody and Anti-αᵥβ₆ Cys-Diabody

Approximately 1×10⁶ 293-F cells were seeded into 6-well plates (Corning) 24 hours prior to transfection in 2 mL of non-selective growth media. On the following day, media was aspirated and cells were transfected with 4 μg of purified pSecTag-B6DiaR-G2S plasmid using Lipofectainine LTX (Invitrogen) in 2 mL, of Opti-MEM (Invitrogen). After 8 hours, transfection reagents were aspirated and wells were replaced with 2 mL of non-selective growth media. Approximately 24 hours later, transfected 293-F cells were expanded into T-75 flasks containing 18 mL of non-selective growth media and cultured for another 24 hours before media was replaced with selective growth media. Transfected cells were grown for approximately 2-3 weeks under selective conditions until stable colonies appeared.

Expression of the anti-αᵥβ₆ diabody and the anti-αᵥβ₆ Cys-diabody from stable mixed pools was analyzed by western blotting to confirm the identity of the constructs. Supernatant samples were removed from each pool and were electrophoresed on a 4-20% Mini-Protean® TGX gel (BioRad) and transferred to a 0.45 μm Immobilon™ polyvinylidene difluoride (PVDF) membrane (Millipore). The membrane was washed twice with Tris-buffered saline (TBS) and blocked with penta-His HRP conjugate blocking buffer (Qiagen) for 1 hour at room temperature. The membrane was washed with TBS/Tween/Triton buffer (2×, 10 minutes), then with TBS buffer (1×, 10 minutes). After washing, the membrane was probed using a Penta-His HRP antibody (diluted 1:2000 in blocking buffer, Qiagen) for 1 h at room temperature followed by washing with TBS/Tween/Triton (2×, 10 mins each) and TBS for 10 minutes each. Detection of the Penta-His HRP antibody was performed using LumiGLO® substrate (Cell Signaling Technology) and the membrane was imaged using a FluorChem M imager (Protein Simple). Stable pools were expanded into T-175 flasks and further into triple flasks (Nunc) for production in approximately 250 mL of production media. Supernatants from triple flasks were harvested and filtered through a 0.2 μm vacuum filter (Nalgene) and stored at 4° C. until purification.

D. Purification of Anti-αᵥβ₆ Diabody and Anti-αᵥβ₆ Cys-Diabody

Approximately 2 mL of His-Pur® nickel nitrilotriacetic acid (Ni-NTA) resin slurry (Thermo Scientific) was equilibrated in binding buffer (PBS, 0.5 M NaCl, 50 mM imidazole, pH 7.5), then transferred to the filtered supernatants (containing either anti-αᵥβ₆ diabody or anti-αᵥβ₆ Cys-diabody). Purification of each diabody was performed overnight at 4° C. on a platform shaker. Supernatants and resins were transferred to 10 mL fritted columns (BioRad) and the flow-through fractions were collected. Resins were washed with binding buffer (10 mL) then eluted in 1 mL fractions with elution buffer (PBS, 500 mM NaCl, 400 mM imidazole, pH 7.5, 6 mL). Elution fractions were electrophoresed on a 4-20% Mini-Protean® TGX gel and stained with PageBlue (Fermentas). Fractions containing purified diabody or Cys-diabody were pooled and dialyzed extensively with dialysis buffer (0.1M HEPES, 0.5M NaCl, 0.1M ethylenediaminetetraacetic acid (EDTA), pH 7.1) and storage buffer (0.1M HEPES, 0.5M NaCl, 10% glycerol, pH 7.1) using Slide-A-Lyzer G2 dialysis cassettes (10 kDa MWCO, Thermo Scientific). Samples were concentrated with centrifugal filters (10 kDa MWCO, Millipore). Final protein concentrations were determined by A280 absorbance using a Nanodrop 2000c (Thermo). The molecular weights and extinction coefficients (51.6 kDa and 106,160 M-1 cm-1 for diabody, 52.1 kDa and 106,285 M-1 cm-1 for Cys-diabody) were calculated from the amino acid sequence using the ExPASy ProtParam webtool.

E. SDS-Page and Size Exclusion Chromatography (SEC)

Purities of the concentrated anti-αᵥβ₆ diabodies and anti-αᵥβ₆ Cys-diabodies were determined using SDS-PAGE according to standard methods known to those skilled in the art. Diabodies were also subjected to size-exclusion chromatography (SEC). Samples containing the purified diabody (50 μg) were loaded onto a Superdex 75 HR 10/300 column (GE Life Sciences) and elution was performed isocratically in PBS at a flow rate of 0.5 mL/min. Elution was monitored at an absorbance of 280 nm and compared to the molecular weight standards of bovine serum albumin (BSA, 66 kDa) and carbonic anhydrase (29 kDa).

F. Western Blotting

To confirm identities of the purified anti-αᵥβ₆ diabody and anti-αᵥβ₆ Cys-diabody, western blots were conducted. Briefly, following SDS-PAGE, the antibody fragments were blotted onto a PVDF membrane. The membrane was blocked with anti-Penta-His HRP conjugate blocking buffer, washed in conjugate buffer, then probed with the anti-Penta-His HRP antibody (1:2000 in blocking buffer). Detection of the anti-Penta-His HRP antibody was done using LumiGLO® substrate and the membrane was imaged using a FluorChem M imager.

G. Electrospray Ionization (ESI)

Purified samples of the anti-αᵥβ₆ diabody or Cys-diabody (10 μL) were injected onto a C8 protein column (Aeris) and eluted with ballistic gradient (12 min) with desalting step (2 min). Data was acquired in positive ion profile mode using the ion trap mass analyzer (LTQ Orbitrap XL, Thermo) scanning from 500-1500 m/z. Data was deconvoluted with MagTran software using standard settings to obtain the parental masses of each antibody fragment.

### Results

A. Expression and Purification of Anti-αᵥβ₆ Diabodies or Anti-αᵥβ₆ Cys-Diabodies

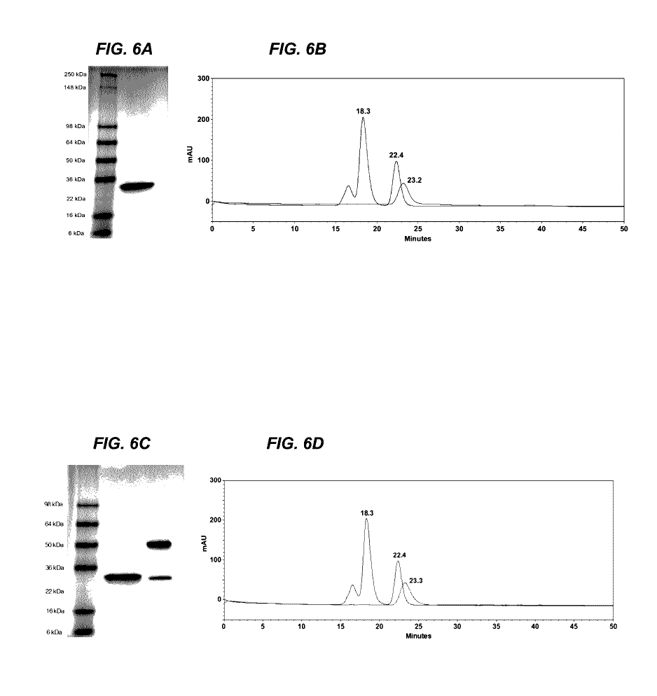

The light and heavy variable chains of the anti-αᵥβ₆ diabody were connected by a flexible three amino acid sequence (G2S), and appended with a C-terminal hexahistadine tag for purification. The construct was expressed in 293-F cells and secreted into the supernatant, which was subsequently filtered and purified by Ni-NTA column chromatography. The purified diabody was electrophoresed under reducing conditions on a 4-20% SDS-PAGE gel and revealed a single band present at ˜25 kDa corresponding to the expected size for the reduced monomeric species with a purity >95% (FIG. 6A). SEC further confirmed the high purity of the anti-αᵥβ₆ diabody by a single absorbance peak at 280 nm with a retention time (TR) of 23.2 mins (FIG. 6B). Interestingly, the purified anti-αᵥβ₆ diabody eluted with a retention time after that of the carbonic anhydrase standard (T_{R}=22.4 mins). This gave the impression that the non-covalent dimer was not being generated during expression, but comparison of the T_{R} of the anti-αᵥβ₆ diabody to that of the covalently bound anti-αᵥβ₆ Cys-diabody confirmed the formation of a dimer. Final product yields of approximately 3 mg/L were obtained from 1 L of supernatant.

The engineered anti-αᵥβ₆ Cys-diabody construct was also successfully expressed using stable mixed pools of 293-F cells under selection with Zeocin. Expression of the Cys-diabody was lower than the parental anti-αᵥβ₆ diabody produced in the same manner, with yields of 1-2 mg/L. The anti-αᵥβ₆ Cys-diabody migrated as a single entity at 25 kDa under reducing SDS-PAGE conditions confirming the high purity obtained from NI-NTA purification. The non-reduced Cys-diabody migrated with a faint band at 25 kDa and a dominant band at 50 kDa (FIG. 6C) confirming primary formation of the covalent dimer.

Samples of the purified anti-αᵥβ₆ Cys-diabody (50 μg) were injected onto the Superdex 75 HR 10/300 SEC column to further confirm size and purity. Retention time was compared to BSA and carbonic anhydrase standards. The formation of the Cys-diabody generated a compact antibody fragment with a retention time (T_{R}) of 23.3 mins, similar to what was observed with the parental anti-αᵥβ₆ diabody (T_{R}=23.2 mins) (FIG. 6D).

To further confirm the sizes of the anti-αᵥβ₆ diabody and Cys-diabody, two other constructs were generated with different linker lengths and the T_{R} of each of these constructs were compared to those of the anti-αᵥβ₆ diabody and Cys-diabody. The B6DiaR-G2S-G4S and B6DiaR-G constructs were constructed and expressed exactly as the parental anti-αᵥβ₆ diabody. The only difference in these constructs is the length of the linker separating the VH and VL domains. The B6DiaR-G2S-G4S construct has a 5 amino acid linker (G4S) and the B6DiaR-G2S-G construct has a 1 amino acid linker (G). From the data it is clear that each of these constructs elutes with nearly identical retention times as the anti-αᵥβ₆ Cys-diabody. Therefore, it was concluded that linker lengths of 1, 3, and 5 amino acids between the V_{H} and V_{L} domains all produce the desired diabody product.

B. Western Blotting

Purified fractions of the anti-αᵥβ₆ diabody and anti-αᵥβ₆ Cys-diabody were electrophoresed on 4-20% precast gels followed by western blotting and detection with the anti-Penta-His-HRP antibody. The anti-αᵥβ₆ diabody was detected as a single band at the expected monomeric weight of 25 kDa. Reduced and non-reduced versions of the anti-αᵥβ₆ Cys-diabody were also detected, and further confirmed construction of the covalent dimer.

C. Electrospray Ionization

Electrospray ionization of the purified anti-αᵥβ₆ Cys-diabody and deconvolution of the data also revealed a main peak corresponding to the expected mass of the covalent dimer at 52,099 Da. This agreed well with the calculated mass of 52,100 Da and confirmed the molecular weight of the anti-αᵥβ₆ Cys-diabody.

### Conclusions

This example provides a method to engineer an anti-αᵥβ₆ diabody and a disulfide-stabilized anti-αᵥβ₆ Cys-diabody which enable non-invasive imaging of αᵥβ₆-positive tumors with PET. Construction of the anti-αᵥβ₆ diabody was based on the variable domains of the humanized 6.3G9 intact anti-αᵥβ₆ antibody and assembled in the VH-G2S-VL format. Similarly, the anti-αᵥβ₆ diabody was constructed in the VH-G2S-VL format, but was engineered with a GGC sequence appended to the C-termini. This feature gives the Cys-diabody an important advantage over the anti-αᵥβ₆ diabody in that the two scFv fragments are covalently bonded, which was hyposthesized to improve the stability and tumor uptake of the fragment in vivo.

The diabodies were successfully generated from stable cell lines. Following purification procedures, the anti-αᵥβ₆ diabody and the disulfide-stabilized anti-αᵥβ₆ Cys-diabody had a purity of >95%. This eliminated the need for a secondary purification step by SEC. The biochemical data showed that the anti-αᵥβ₆ diabody forms a non-covalent dimer.

Utilizing the humanized antibody platform described herein as the basis for construction of these molecular imaging agents shall be highly beneficial with regards to clinical translation as it virtually eliminates the human anti-mouse antibody (HAMA) response observed from antibodies of murine origin.

### Example 2

### Biochemical Characterization of Anti-αᵥβ₆ Integrin Diabodies and Disulfide-Stabilized Anti-αᵥβ₆ Integrin Diabodies and Generation of Radiolabeled Anti-αᵥβ₆ Integrin Diabodies and Disulfide-Stabilized Anti-αᵥβ₆ Integrin Diabodies

This example illustrates the binding affinity and selectivity of the anti-αᵥβ₆ integrin diabodies and disulfide-stabilized anti-αᵥβ₆ integrin diabodies for αᵥβ₆ integrin.

A competitive binding enzyme-linked immunosorbant assay (ELISA) was used to determine binding affinity of each diabody towards immobilized αᵥβ₆ integrin relative to fibronectin, the natural binding ligand of αᵥβ₆. Prior work has demonstrated that obtaining a subnanomolar binding affinity towards the target antigen is highly desirable for pursuing in vivo preclinical imaging studies. To determine selectivity of the diabodies for the αᵥβ₆ integrin, two human melanoma cell lines were used, one of which has been engineered to overexpress the αᵥβ₆ integrin. Flow cytometry was performed on the DX3Puroβ₆ (αᵥβ₆+) and the DX3Puro (αᵥβ₆−) cell lines, using the anti-αᵥβ₆ diabody and anti-αᵥβ₆ Cys-diabody as primary reagents for the detection αᵥβ₆.

To convert the anti-αᵥβ₆ diabody and anti-αᵥβ₆ Cys-diabody into PET imaging agents the anti-αᵥβ₆ diabody and anti-αᵥβ₆ Cys-diabody were radiolabeled on the 6-amines of exposed lysine residues using the activated ester N-succinmidyl-4-[¹⁸F]-fluorobenzoate ([¹⁸F]-SFB]) in a non-site-specific manner. A thorough evaluation of the radiochemical yields and radiochemical purities of each of the [¹⁸F]-radiolabeled diabodies was performed, followed by cell binding assays on DX3Puroβ₆ and the DX3Puro cell lines to determine what effects this radiolabeling approach had on immunoreactivity of each PET imaging agent. The introduction of [18F]-SFB onto the diabodies was performed in a non-site-specific manner.

A site-specific radiolabeling approach was also conducted on the anti-αᵥβ₆ Cys-diabody using [⁶⁴Cu]. To incorporate [⁶⁴Cu], the bifunctional chelator (BFC) 1,4,7-triazacyclononane-1,4,7-triacetic acid (NOTA) was used. Though [⁶⁴Cu] is the primary radioisotope used with this BFC, a range of radioisotopes have been successfully incorporated onto biomolecules with NOTA including [⁶⁸Ga] (t_{1/2}=271 days), [¹¹¹In] (t_{1/2}=2.8 days), and [¹⁸F] (t_{1/2}=109.7 mins), thus enabling tremendous flexibility in terms of choice of radioisotope and imaging modality. In this manner, by using a employing a versatile BFC, a flexible platform was created in which other diabody-based molecular imaging agents can be generated and characterized. As with its [¹⁸F]-radiolabeled counterparts, the immunoreactivity of the [⁶⁴Cu]-NOTA-αᵥβ₆ Cys-diabody was assessed through cell binding assays on DX3Puroβ₆ and the DX3Puro cell lines.

### Materials and Methods

A. Cell Lines and Cell Culture Reagents

The DX3Puro and DX3Puroβ₆ cell lines were cultured in DMEM supplemented with 10% FBS and 1% PSG. The DX3Puroβ₆ cell line was generated by retroviral transfection with human β₆ cDNA as previously described. All cell lines were cultured in cell culture T-flasks (Greiner Bio-One) at 37° C. in a humidified incubator with 5% CO₂ unless otherwise noted.

B. Competitive Binding Enzyme-Linked Immunosorbant Assay (ELISA)

Separate competitive binding ELISAs were used to determine the binding affinity of the anti-αᵥβ₆ diabody and Cys-diabody relative to biotinylated fibronectin, a known natural ligand of the αᵥβ₆ integrin. Biotinylation of fibronectin was performed according to the manufacturer's instructions (Amersham Biosciences). The P2W7 antibody (anti-αᵥ, 5 μg/mL, Abcam) was plated into wells (50 μL/well) of a 96-well plate (Maxisorp®, Nunc). The plate was incubated at 37° C. for 1 h then washed with PBS (3×). After washing, the wells were blocked with blocking buffer (PBS, 5% bovine serum albumin (BSA), 1% Tween 20) and the plate was incubated at room temperature for 3 hrs, followed by washing with PBS (3×). The αᵥβ₆ integrin (3 μg/mL, R&D Systems) in conjugate buffer (20 mM Tris, 1 mM MnCl2, 150 mM NaCl, 0.1% Tween, 1% BSA) was added to the wells (50 μL/well). The plate was incubated at room temperature for 1 h and washed with wash buffer (20 mM Tris, 1 mM MnCl2, 150 mM NaCl, 0.1% Tween, 3×). Competitive binding to the αᵥβ₆ integrin was conducted between serial dilutions of the anti-αᵥβ₆ diabody or Cys-diabody (1 μM 0.5 μM in conjugate buffer) and biotinylated fibronectin (50 μL/well). The plate was incubated at room temperature for 1 h and washed with wash buffer (3×). Extravidin HRP conjugate (Sigma Aldrich) was added to each well (50 μL/well) and the plate was incubated at room temperature for 1 h, followed by washing in wash buffer (3×). Extravidin HRP conjugate was detected using 3,3′,5,5′-tetramethylbenzidine (TMB) substrate (50 μL/well) for 10-15 mins at room temperature. The reaction was terminated by the addition of 1N sulfuric acid (50 μL/well) and the absorbance of each well was measured in a Multiscan Ascent plate reader (Thermo) at 450 nm. Wells containing biotinylated fibronectin served as the positive control. Two negative controls were used. The first negative control was lacking the P2W7 antibody and diabody (or Cys-diabody). The second negative control was lacking biotinylated fibronectin and diabody (or Cys-diabody).

C. Flow Cytometry

DX3Puro and DX3Puroβ₆ cells were harvested at 70-90% confluency and counted using trypan blue exclusion. Cells were washed in wash buffer (PBS, 0.1% BSA, 0.1% sodium azide, 3×) and resuspended in wash buffer to a concentration of 3×105 cells per 50 pt. Aliquots of the cell suspension (50 μL) were transferred to 1.5 mL microcentrifuge tubes. The anti-αᵥβ₆ antibody 10D5 (Millipore), IgG2a isotype control antibody (Biolegend), and anti-αᵥβ₆ diabody were diluted to 10 μg/mL in serum-free DMEM (Gibco) and incubated separately with each cell suspension for 1 h at 4° C. Cells were washed with wash buffer (3×) and the 10D5 and IgG2a antibodies were detected with AlexaFluor488 goat anti-mouse secondary antibody (10 μg/mL, Invitrogen) for 30 mins at 4° C. The anti-αᵥβ₆ diabody was detected with the anti-Penta-His AlexaFluor488 conjugate (10 μg/mL, Qiagen). Cell were washed with wash buffer (3×), resuspended in 500 μL of wash buffer, and transferred to 5 mL polystyrene tubes (BD Falcon). Fluorescent signal was acquired with a FC500 FACS machine (Beckman Coulter) and data was analyzed using FlowJo X (TreeStar, Inc.). This process was repeated in its entirety for the anti-αᵥβ₆ Cys-diabody.

D. Radiolabeling of Anti-αᵥβ₆ Diabody and Anti-αᵥβ₆ Cys-Diabody with N-Succinimidyl-4-[¹⁸F]-Fluorobenzoate ([¹⁸F]-SFB)

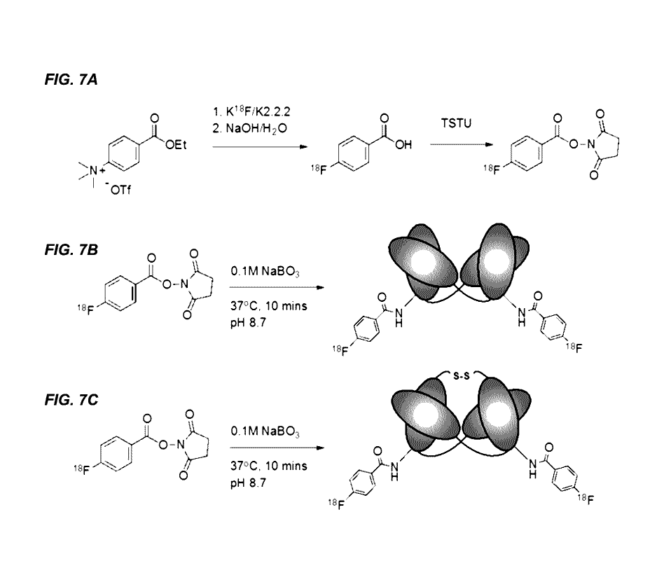

Automated synthesis of [¹⁸F]-SFB was conducted according to a previously published procedure with the addition of high performance liquid chromatography (HPLC) purification (FIG. 7A). Dried [¹⁸F]-SFB was reconstituted into a 1 mL solution of 0.05% trifluoroacetic acid in water and acetonitrile (1:1 v/v). The solution was injected onto a Beckman-Coulter (Brea, Calif.) HPLC system equipped with a Jupiter Proteo C-18 semi-preparative column (10 μM, Phenomenex) at a flow rate of 3.0 mL/min. Mobile phase consisted of a linear gradient of solvent A (0.05% trifluoroacetic acid in water) (9%) and solvent B (100% acetonitrile), beginning with 9% solvent B and increasing to 81% solvent B over 30 mins. The radioactive fraction was collected and trapped onto a pre-conditioned C18 Sep-Pak Plus cartridge (Waters). The cartridge was washed with deionized water (5 mL), dried with air, and eluted with dichloromethane (DCM, 2 mL).

The anti-αᵥβ₆ diabody in 0.1M HEPES, 0.5M NaCl, pH 7.1 (200-300 μg) was diluted 1:20 with 20× borate buffer. An aliquot of [¹⁸F]-SFB (50-60 mCi) in DCM was dried under a stream of nitrogen and reconstituted in 50 μL of 1× borate buffer, pH 8.7. The anti-αᵥβ₆ diabody (150 μL) was immediately added to the dissolved [¹⁸F]-SFB and the reaction was incubated at 37° C. for 10 mins (FIG. 7B). The reaction volume was transferred to a BioSpin P-30 column (40 kDa MWCO, BioRad) pre-equilibrated with 1% saline and the column was measured in a dose calibrator (CRC-15R, Capintec). The [¹⁸F]-SFB labeled anti-αᵥβ₆ diabody was eluted from the column by centrifugation and the radioactivity of the eluted fraction was measured. Radiochemical yields (RCY) were calculated based on radioactivity of eluted [¹⁸F]-FB-αᵥβ₆ diabody divided by the total radioactivity applied to the spin column. To confirm radiochemical purity (RCP), a sample of the eluted fraction of the [¹⁸F]-FB-αᵥβ₆ diabody was injected onto a Waters radio-HPLC equipped with a Superdex 75 HR 10/300 column and photomultiplier tube (PMT) ran isocratically in 0.1M NaPO3, pH 6.8 at 0.5 mL/min. Radiolabeling of the anti-αᵥβ₆ Cys-diabody with [¹⁸F]-SFB was conducted as described above (FIG. 7C).

E. Site-Specific Radiolabeling of the Anti-αᵥβ₆ Cys-Diabody with [⁶⁴Cu]

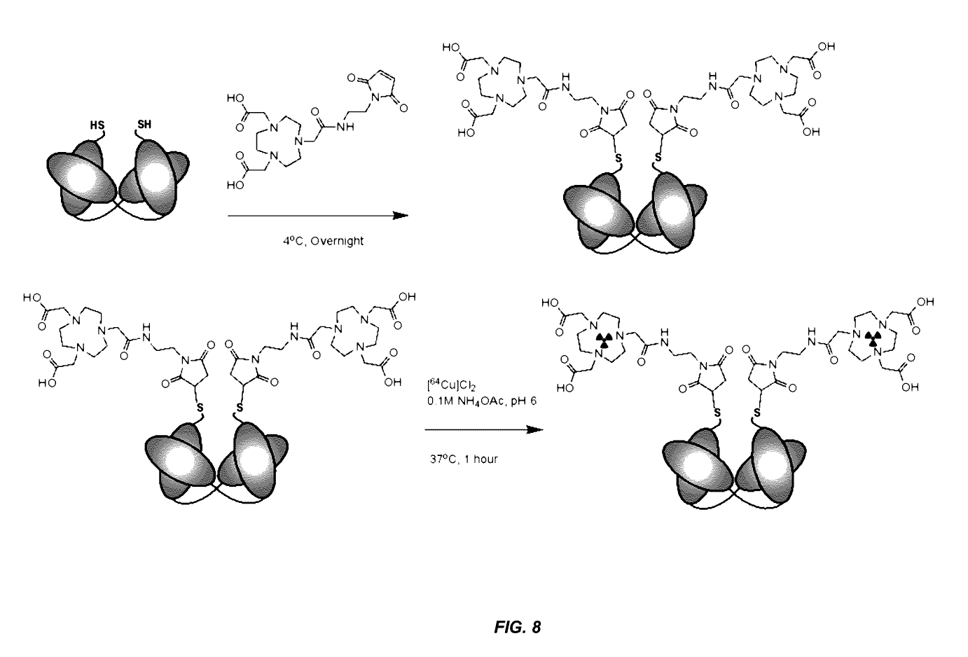

FIG. 8 illustrates the conjugation of NOTA-maleimide and subsequent radiolabeling of the anti-αᵥβ₆ Cys-diabody with [⁶⁴Cu]. The anti-αᵥβ₆ Cys-diabody was reduced with tris(2-carboxyethyl)phosphine (TCEP) Bond Breaker™ (25 mM, Pierce). The reaction was mixed and allowed to proceed at 4° C. for 30 mins. NOTA-maleimide (0.57 mg, 0.84 μmol, Chematech) was dissolved in DMSO and added directly to the reduced Cys-diabodies. The conjugation reaction proceeded for 5 hrs at 4° C. with occasional shaking. Excess chelator was removed with a PD-10 column (GE Life Sciences) pre-equilibrated with chelex treated storage buffer. Fractions containing NOTA-conjugated Cys-diabody were pooled and reconcentrated using centrifugal filters (Amicon, 10K MWCO).

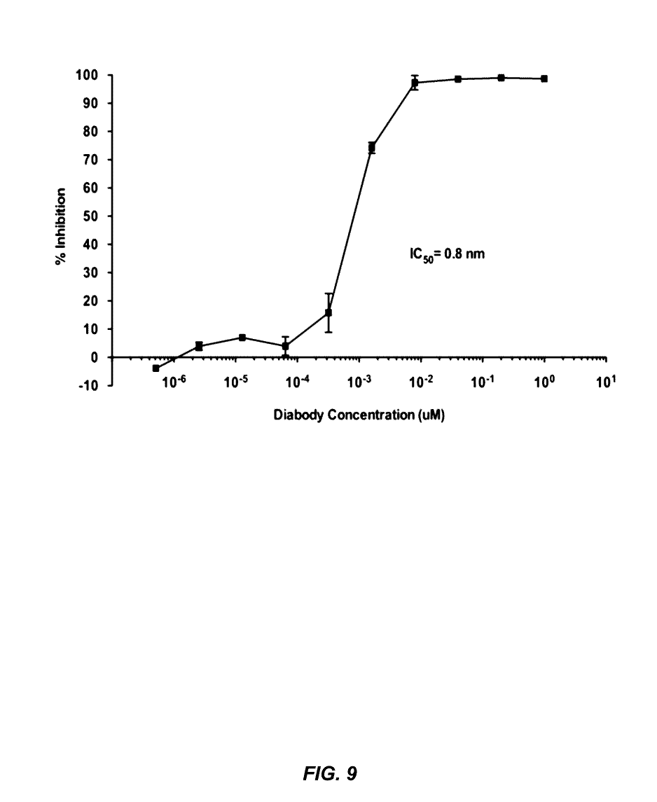

The [⁶⁴Cu] was obtained in the form of [⁶⁴Cu]Cl₂ (3-8 mCi) in 0.5 M HCl (5-10 μL). It was subsequently diluted in chelex treated 0.1 M NH4OAc, pH 7. The NOTA-αᵥβ₆ Cys-diabody (1.7 mg/mL, 40 μL) was buffer-exchanged into chelex treated 0.1 M NH₄OAc, pH 7 using a BioSpin P-30 column (BioRad) and added directly to [⁶⁴Cu]OAc (1-3 mCi, 4-12 μL). The reaction was incubated at 40° C. for 1 h (FIG. 9). Radiochemical yield (RCY) of the crude reaction was determined using antibody instant thin layer chromatography strips (iTLC, Biodex). Briefly, 1-2 μL of the reaction was spotted on iTLC strips, which were subsequently placed in mobile phase (PBS, 90 mM EDTA, pH 7) and allowed to migrate to the top solvent line. The strip was analyzed with an AR-2000 radio-TLC imager (Biodex) and radioactive peaks were integrated. The crude reaction was challenged with 0.1 M NH₄OAc, 5 mM EDTA, pH 7 for 15 mins at room temperature and purified using a Biospin-30 column. Final radiochemical yield (RCY) was determined by dividing the radioactivity eluted from the spin column by the initial amount of radioactivity applied to the spin column. Radiochemical purity (RCP) of the purified [⁶⁴Cu]-NOTA-αᵥβ₆ Cys-diabody was confirmed by radio-HPLC using a Superdex 75 column connected in series to a photomultiplier tube (PMT) using 0.1M NaPO₃, pH 6.8 as the mobile phase at a flow rate of 0.5 mL/min.

F. Immunoreactivity

DX3Puroβ₆ and DX3Puro cell lines were used as positive and negative controls, respectively. Cells were harvested by trypsinization and counted using trypan blue exclusion. Microcentrifuge tubes (1.5 mL, USA Scientific) were blocked with blocking buffer (5% BSA in PBS) for 15 mins at room temperature and washed with serum-free DMEM (2×). Separate cell suspensions (50 μL, 3.75×106 cells) were combined either with the [¹⁸F]-FB-αᵥβ₆ diabody or the [¹⁸F]-FB-αᵥβ₆ Cys-diabody (50 μL, 0.2 μCi) and incubated at room temperature for 1 h with gentle shaking. Cell suspensions were pelleted by centrifugation, washed with serum-free DMEM, and the radioactivity present in the pellets was compared to the radioactivity present in the supernatant fractions using a gamma counter (Wizard 1470, Perkin Elmer). Internalization of each antibody fragment was determined by incubating each of the cell pellets with cold acid solution (300 μL, 0.2 M sodium acetate, 0.2 M sodium chloride, pH 2.5) at 4° C. for 5 mins. Cell suspensions were centrifuged and the supernatants were collected. Pellets were washed with PBS (300 μL), centrifuged again, and the wash fractions were combined with previous acid supernatants. Radioactivities in the pellet and wash fractions were analyzed as described above. Immunoreactivities and percent internalization for each antibody fragment were calculated by dividing the radioactivity in the respective pellets by the total radioactivity in the wash and pellet fractions. A separate set of identical immunoreactivity experiments was performed exactly as described above for the site-specifically radiolabeled [⁶⁴Cu]-NOTA-αᵥβ₆ Cys-diabody.

### Results

Competitive binding ELISA against fibronectin, a known natural ligand of the αᵥβ₆ integrin, was used to assess the binding activity of the anti-αᵥβ₆ diabody. Biotinylated fibronectin was mixed 1:1 with serial dilutions of anti-αᵥβ₆ diabody and allowed to compete for binding to immobilized αᵥβ₆ integrin. FIG. 9 demonstrates the binding activity of the anti-αᵥβ₆ diabody relative to the biotinylated fibronectin competitor after absorbance was read at 450 nm. Sigmoidal curve fitting revealed highly potent binding of the anti-αᵥβ₆ diabody with an IC50=0.8 nM.

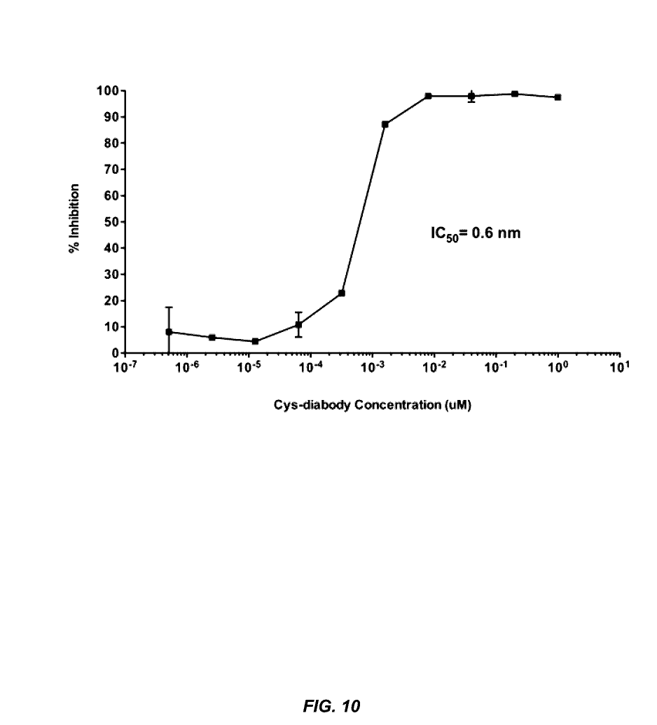

Similarly, after competitive binding towards αᵥβ₆ between serial dilutions of the anti-αᵥβ₆ Cys-diabody and fibronectin, a sigmoidal curve was fit to the chemiluminescent absorbance data (FIG. 10). The calculated IC50 of the anti-αᵥβ₆ Cys-diabody was 0.6 nM, indicating potent binding to the αᵥβ₆ integrin. This is a slight improvement in binding affinity over the parental anti-αᵥβ₆ diabody and supports the fact that introduction of the engineered disulfide bond did not detrimentally affect binding affinity towards the αᵥβ₆ integrin.

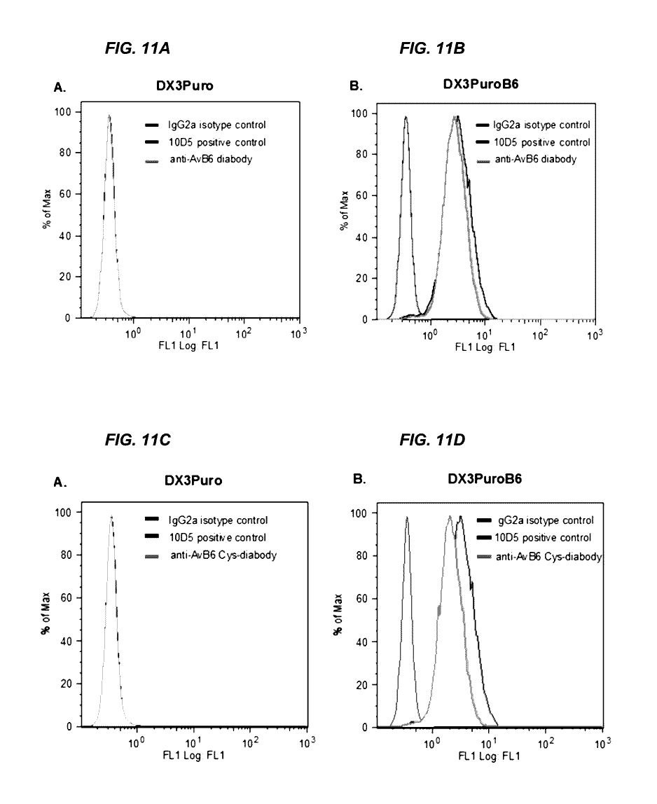

To determine binding specificity of the anti-αᵥβ₆ diabody to the αᵥβ₆ integrin, flow cytometry was performed on the DX3Puro and DX3Puroβ₆ cell lines (FIGS. 11A and B). When the isotype IgG2A control was incubated with each cell line, no shift was observed in the fluorescent intensity. As a positive control, the 10D5 antibody was incubated with each cell line, generating a shift in fluorescent intensity only in the DX3Puroβ₆ cell line, indicating specific binding to the αᵥβ₆ integrin. Similar to the 10D5 control antibody, a strong shift in fluorescent signal was observed only when the anti-αᵥβ₆ diabody was incubated with the DX3Puroβ₆ cell line, confirming specific binding to the αᵥβ₆ integrin.

FIGS. 11C and D illustrate the results from flow cytometry experiments conducted on DX3Puro and DX3Puroβ₆ cell lines with the anti-αᵥβ₆ Cys-diabody. Similar to the parental anti-αᵥβ₆ diabody, a shift in fluorescent signal at 488 nm was observed only for the DX3Puroβ₆ cell line indicating specific binding to the αᵥβ₆ integrin.

Synthesis and HPLC purification of [¹⁸F]-SFB was performed with a decay-corrected radiochemical yield of 36.1%±2.3% and a synthesis time of 63.3±1.0 mins. Radiochemical purity (RCP) was >98% as determined from radio-HPLC. Non-site specific radiolabeling of the anti-αᵥβ₆ diabody with HPLC purified [¹⁸F]-SFB was accomplished within 10 mins with a radiochemical yield (RCY) of 22.6%±3.6%. These yields exceeded RCYs of diabodies manually radiolabeled with [¹⁸F]-SFB, and closely matched those reported after [¹⁸F]-SFB labeling of diabodies was optimized with microfluidics. Spin-column purification separated unreacted [¹⁸F]-SFB from the radiolabeled [¹⁸F]-FB-αᵥβ₆ diabody and resulted in a radiochemical purity >98% with no additional purification necessary. Following purification, the calculated specific activity was ˜550 Ci/mmol.

The anti-αᵥβ₆ Cys-diabody was radiolabeled with [¹⁸F]-SFB within 10 mins with RCY of 8.3±1.7%. The RCY of the [¹⁸F]-FB-αᵥβ₆ Cys-diabody was lower than that observed for the parental [¹⁸F]-FB-αᵥβ₆ diabody, but still resulted in ample amounts of radiolabeled compound needed for further studies. Spin-column purification separated unreacted [¹⁸F]-SFB from radiolabeled FB-αᵥβ₆ Cys-diabody and resulted in radiochemical purity >98% with no additional purification necessary. Specific activity ranged between 400-500 mCi/μmol.

For site-specific radiolabeling of the anti-αᵥβ₆ Cys-diabody with [⁶⁴Cu], The anti-αᵥβ₆ Cys-diabody were reduced and conjugated to NOTA-maleimide to enable radiolabeling with [⁶⁴Cu]. Radiolabeling with [⁶⁴Cu] was accomplished in 1 h at 40° C. with crude radiochemical yields >93% by iTLC (n=3). Final radiolabeling yields of the [⁶⁴Cu]-NOTA-αᵥβ₆ Cys-diabody were 34.1±7.3% (n=3) following spin-column purification. Since separation of the unlabeled Cys-diabody is practically difficult from the [⁶⁴Cu]-labeled Cys-diabody, the effective specific activity (ESA) was calculated by dividing the original radioactivity in the crude reaction by the total mass of Cys-diabody in the reaction. This value was corrected by multiplying the ESA by the final radiochemical yield of the [⁶⁴Cu]-NOTA-αᵥβ₆ Cys-diabody following spin column purification. Radiochemical purity of the j NOTA-αᵥβ₆ Cys-diabody was >95% following spin column purification as determined by SEC. The calculated ESA of the [⁶⁴Cu]-NOTA-αᵥβ₆ Cys-diabody was between 450-550 mCi/μmol.

Specificity and immunoreactivity were well preserved after [¹⁸F]-SFB labeling with 58.7±6.7% of the radiolabeled diabody bound to the DX3Puroβ₆ cell line, compared to only 2.5±0.1% binding to the DX3Puro cell line (n=3). Total internalization of the [¹⁸F]-FB-αᵥβ₆ diabody was found to be 44.4±2.1% for the DX3Puroβ₆ cell line and only 1.6±0.5% for the DX3Puro cell line.

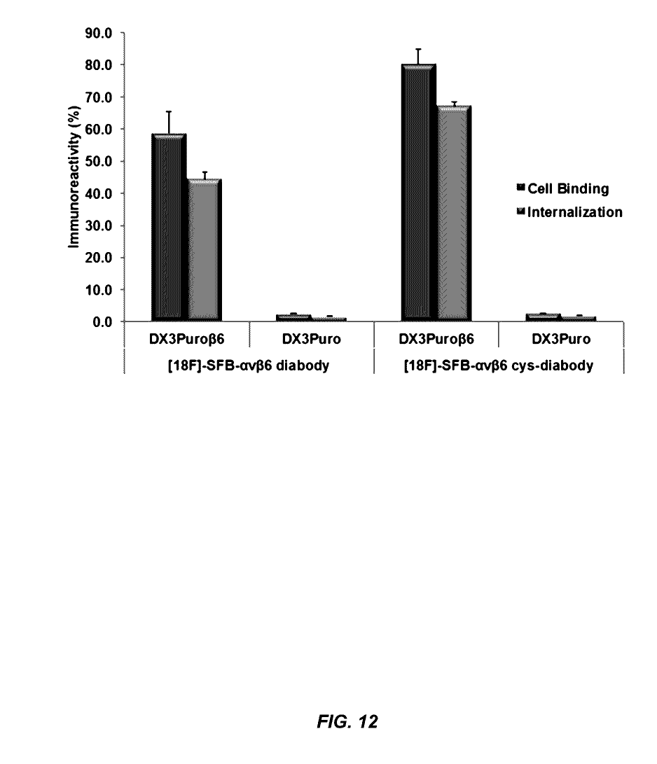

In vitro cell binding experiments demonstrated excellent preservation of immunoreactivity and specificity after [¹⁸F]-SFB radiolabeling of the anti-αᵥβ₆ Cys-diabody (FIG. 12). Approximately 80.4±4.4% of the radioactivity remained bound to the DX3Puroβ₆ cell line compared to only 2.6±0.1% bound to the DX3Puro cell line. Internalization of the [¹⁸F]-FB-αᵥβ₆ Cys-diabody was found to be 67.0±1.3% on the DX3Puroβ₆ cell line, whereas only 1.8±0.1% of the radioactivity was internalized for the DX3Puro cell line.

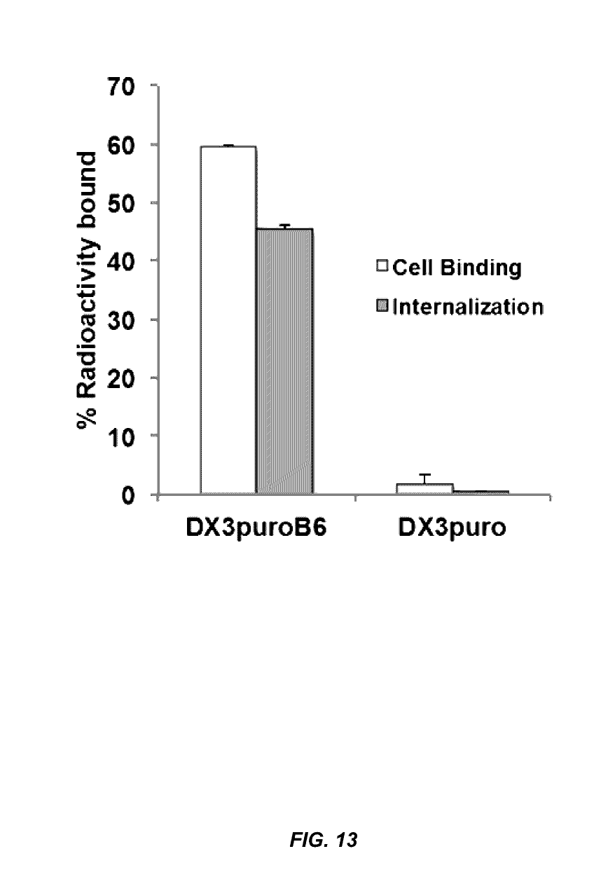

Immunoreactivity was also well-preserved following site-specific radiolabeling with [⁶⁴Cu] with approximately 60% of the [⁶⁴Cu]-NOTA-αᵥβ₆ Cys-diabody demonstrating selective cell binding to the DX3Puroβ₆ cell line, with less than 2% bound to the DX3Puro line (FIG. 13). Approximately 45% of the [⁶⁴Cu]-NOTA-αᵥβ₆ Cys-diabody was internalized in the DX3Puroβ₆ within the 1 h incubation time, whereas less than 1% was internalized in the αᵥβ₆-negative DX3Puro cell line.

### Conclusions

Functional characterization of the anti-αᵥβ₆ diabody and anti-αᵥβ₆ Cys-diabody was demonstrated via competitive binding ELISA and flow cytometry to confirm affinity and specificity towards αᵥβ₆, respectively. The anti-αᵥβ₆ diabody demonstrated potent subnanomolar binding affinity towards the αᵥβ₆ integrin with highly specific binding to the DX3Puroβ₆ cell line. Similarly, the anti-αᵥβ₆ Cys-diabody revealed nearly identical binding affinity and specificity, further confirming that the addition of the engineered disulfide bond had no detrimental effects on binding affinity or antigen recognition. These results further demonstrate the flexibility of the diabody platform and its tolerance of minor sequence additions without deleterious effects to the binding characteristics.

To convert the diabodies into molecular imaging agents for PET, radiolabels were added site-specifically or non-site specifically to the diabodies. For instance, the activated ester [¹⁸F]-SFB was conjugated to 6-amines of exposed lysine residues.

In vitro cell binding experiments with the [¹⁸F]-FB-αᵥβ₆ diabody and [¹⁸F]-FB-αᵥβ₆ Cys-diabody on DX3Puroβ₆ and DX3Puro cell lines demonstrated high retention of immunoreactivity and selectivity towards the DX3Puroβ₆ cell line.

### Example 3

### In Vivo Characterization of Radiolabeled Anti-αᵥβ₆ Integrin Diabodies and Disulfide-Stabilized Anti-αᵥβ₆ Integrin Diabodies

This example describes a study of the tumor uptake and pharmacokinetics of the [¹⁸F]-FB-αᵥβ₆ diabodies and the [¹⁸F]-FB-αᵥβ₆ Cys-diabodies described above. The example also describes the in vivo targeting characteristics and pharmacokinetics of the site-specifically radiolabeled [⁶⁴Cu]-NOTA-αᵥβ₆ Cys-diabody.

Briefly, small animal PET and CT imaging were conducted for each of the three PET imaging agents, followed by comprehensive ex vivo analysis from biodistribution, autoradiography, and immunohistochemical (IHC) staining. Following injection of the [¹⁸F]-FB-αᵥβ₆ diabody and the [¹⁸F]-FB-αᵥβ₆ Cys-diabody into separate groups of tumor-bearing mice, small animal PET and CT imaging was conducted at 1, 2, 4, and 6 h post-injection (p.i.). Separate groups of mice were also subjected to ex vivo biodistribution at 1, 2, 4 and 6 h p.i. while autoradiography and IHC staining were only done at 6 h p.i. Similar in vivo and ex vivo experiments were conducted for the [⁶⁴Cu]-NOTA-αᵥβ₆ Cys-diabody with the main difference being the acquisition of PET and CT data at 4, 12, and 24 h timepoints, with biodistribution and IHC only occurring immediately after the 24 h timepoint.

### Materials and Methods

A. Animal Model

All animal handling and procedures were performed using protocols approved by the UC Davis Institutional Animal Care and Use Committee. Approximately 2-3 weeks before PET imaging, 6-8 week old athymic female nu/nu mice (Charles River Laboratories) were injected subcutaneously in opposite shoulders with DX3Puro and DX3Puroβ₆ cells (3×10⁶ cells/line) in 100 μl serum-free DMEM. Tumors were allowed to grow to 0.5-1 cm final diameter prior to conducting imaging and biodistribution experiments.

B. Small Animal PET/CT Imaging

The [¹⁸F]-SFB radiolabeled diabodies (˜210 μCi, 25 μg for [¹⁸F]-FB-anti-αᵥβ₆ diabody; ˜240 μCi, 23 μg for [¹⁸F]-FB-anti-αᵥβ₆ Cys-diabody) were formulated in 1% sterile saline, was injected via catheter into the tail vein of mice anesthetized with 2-3% isoflurane. A fifteen-minute uptake period was allowed for the radiotracer prior to PET imaging. Two mice were imaged simultaneously in the prone position on a dedicated Inveon microPET scanner (Siemens Molecular Imaging) while maintaining 1.5-2% isoflurane anesthesia. A 1 h dynamic scan was followed by 15 min static scans at 2, 4, and 6 h post-injection (p.i.). Immediately after each PET scan, a 10 min cobalt-57 attenuation correction scan was acquired, followed by a 15 min CT scan for anatomical reference. [¹⁸F]-filled capillaries were used as fudicial markers to enable co-registration of PET and CT data. PET images were reconstructed using ordered-subsets expectation maximization 3D maximum a posteriori algorithms (OSEM3D/fastMAP) and co-registered with CT images using Inveon Research Workstation software.

Small animal PET and CT imaging for the [⁶⁴Cu]-NOTA-αᵥβ₆ Cys-diabody were conducted as described above. The [⁶⁴Cu]-NOTA-αᵥβ₆ Cys-diabodies (˜220 μCi, 10.6 μg) were formulated in 1% saline and doses were administered intravenously via tail vein into each mouse. Mice were scanned using an Inveon microPET scanner (Siemens Molecular Imaging) for 15 mins at 4 and 12 h p.i. and for 30 mins at 24 h p.i. A 10 min cobalt-57 attenuation correction scan was performed after each PET scan followed by a 15 min CT scan (Inveon, Siemens Molecular Imaging) for anatomical reference. PET images were reconstructed using ordered-subsets expectation maximization 3D maximum a posteriori algorithms (OSEM3D/fastMAP) and co-registered with CT images using Inveon Research Workstation software.

C. Biodistribution

For imaging studies in which the [¹⁸F]-FB-anti-αᵥβ₆ diabody or [¹⁸F]-FB-anti-αᵥβ₆ Cys-diabody were being evaluated, mice were anesthetized with 2-3% isoflurane and the radiotracers were injected into separate sets of mice via tail vein catheter. Mice that were injected with the [¹⁸F]-FB-αᵥβ₆ diabody received ˜46 μCi (3 μg). Similarly, mice that were injected with the [¹⁸F]-FB-αᵥβ₆ Cys-diabodies also received ˜46 μCi (3.6 μg). At 2, 4, and 6 h p.i, mice (3 mice/time point) were sacrificed and dissected. Organs, tissues, and tumors were removed and weighed, and radioactivity was measured with a gamma counter. Radioactivity was calculated as % ID/g based on decay-correction of the administered dose. Statistical analysis was done using paired two-tailed Student's t tests with p<0.05 considered to be statistically significant.

Mice that received injections of the [⁶⁴Cu]-NOTA-αᵥβ₆ Cys-diabodies for small animal PET and CT imaging were euthanized immediately following the final CT scan at 24 h p.i and dissected. Gamma counting, % ID/g calculations, and statistical significance were calculated as described above.

D. Immunohistochemistry (IHC) and Autoradiography

Separate mice were administered injections of the [¹⁸F]-FB-αᵥβ₆ diabody (˜1 mCi, 61 μg) or the [¹⁸F]-FB-αᵥβ₆ Cys-diabody (˜1 mCi, 81 μg). Tumors were excised at 6 h p.i and immediately embedded in freezing medium for sectioning. The tumors were sliced using a cryotome and either stored frozen on microscope slides (5 μM thickness) or exposed to a storage phosphor screen overnight (20 μM thickness). The following day, a phosphor imager was used to read autoradiography samples at a 50 μM pixel resolution. Immunohistochemical analysis was used to detect the presence of the αᵥβ₆ integrin and the anti-αᵥβ₆ diabody (or Cys-diabody). Slides were fixed with periodate-lysine-paraformaldehyde solution and washed with tris-buffered saline with Tween-20 (TBST). Endogenous peroxidase was blocked using 3% hydrogen peroxide in 0.3% horse serum in TBS for 5 mins at room temperature, followed by washing with TBS. Non-specific binding was blocked with 2.5% horse serum for 20 mins. The αᵥβ₆ integrin was detected using a goat anti-β₆ antibody (Vector Labs) for 1 h, with a PBS negative control. After washing with TBS, a secondary anti-goat-HRP-labeled IgG (Vector Labs) was incubated for 30 mins. The anti-αᵥβ₆ diabody (or Cys-diabody) was detected with the goat anti-Penta-His antibody (Thermo), followed by the HRP-conjugated anti-goat secondary antibody (Immpress, Vector Labs). Staining was developed with ImmPACT DAB substrate (Vector Labs) and counterstained with Mayer's hematoxylin. Slides were mounted with DPX mountant (Sigma) and digitally archived.

A separate mouse was administered an injection of the [64Cu]-NOTA-αᵥβ₆ Cys-diabody (˜1 mCi, 60 μg). At 24 h p.i., tumors were excised and immediately embedded in freezing medium for sectioning. Tumor slicing and staining was performed exactly as described above, followed by digital archiving.

E. Data Analysis

All values are reported as mean±standard deviation. Paired two-tailed Student's t tests were used to determine statistical significance, with p<0.05 considered to be statistically significant.

### Results

A. Small Animal In Vivo PET Imaging and Ex Vivo Biodistribution of [¹⁸F]-FB-αᵥβ₆ Diabody

To visualize the tumor targeting properties of the [¹⁸F]-FB-αᵥβ₆ diabody in vivo, mice bearing DX3Puroβ₆ (left shoulder) and DX3Puro (right shoulder) tumor xenografts were administered intravenous doses of the diabody (˜210 μCi, 25 μg). The average tumor mass was 82.2±72.1 mg for the DX3Puro tumors and 43.3±13.3 mg for the DX3Puroβ₆ tumors. A total of four mice were scanned following tail vein injection of spin-column purified [¹⁸F]-FB-αᵥβ₆ diabody. Small animal PET imaging revealed tumor contrast as early as 2 h p.i. As expected with an antibody fragment of this size, primary clearance was observed through the renal pathway in all mice with increased PET signal observed in the kidneys. This observation was well correlated with biodistribution with high kidney uptake measured via gamma counting. Blood clearance of the [¹⁸F]-FB-αᵥβ₆ diabody rapidly decreased between the 2 and 6 h time points yet there was a persistent uptake in the blood pool and heart after 6 h (1.47±0.25% ID/g blood, 1.90±0.21% ID/g heart).

B. Small Animal In Vivo PET Imaging and Ex Vivo Biodistribution of [¹⁸F]-SFB-αᵥβ₆ Cys-Diabody

To determine the in vivo tumor targeting characteristics of the [¹⁸F]-FB-αᵥβ₆ Cys-diabody, doses of the radiotracer containing 240 μCi of radioactivity (˜23 μg of protein) were injected intravenously into the tail veins of male nu/nu murine models bearing DX3Puroβ₆ and DX3Puro tumor xenografts. The average tumor mass was 94.4±42.1 mg for the DX3Puro tumors and 67.3±31.6 mg for the DX3Puroβ₆ tumors. Small animal PET imaging revealed tumor contrast as early as 2 h p.i. Tumor contrast continued to increase through the 6 h time point, as blood levels of the [¹⁸F]-FB-αᵥβ₆ Cys-diabody decreased over time. Ex vivo biodistribution revealed an increase in tumor uptake for the DX3Puroβ₆ tumors when compared to the DX3Puro tumors. The overall uptake of the [¹⁸F]-FB-αᵥβ₆ Cys-diabody in the tumors was considerably higher than that observed for the parental [¹⁸F]-FB-αᵥβ₆ diabody at each time point. For example, at 6 h p.i., the DX3Puroβ₆ tumors contained approximately 1.5% ID/g of the [¹⁸F]-FB-αᵥβ₆ diabody, which increased to approximately 2.5% ID/g for the [¹⁸F]-FB-αᵥβ₆ Cys-diabody. Kidney uptake of the [¹⁸F]-FB-αᵥβ₆ Cys-diabody was persistent throughout each of the time points and clearly visible, but had also increased by approximately 50% over the parental [¹⁸F]-FB-αᵥβ₆ diabody to 34% ID/g.

C. Small Animal In Vivo PET Imaging and Ex Vivo Biodistribution of [⁶⁴Cu]-NOTA-αᵥβ₆ Cys-Diabody

To assess the in vivo performance of the [⁶⁴Cu]-NOTA-αᵥβ₆ Cys-diabody, the molecular imaging agent was injected intravenously into the tail vein of mice bearing DX3Puroβ₆ (left shoulder) and DX3Puro (right shoulder) tumor xenografts and subsequently imaged with PET. The average tumor mass was 83.9±12.3 mg for the DX3Puro tumors and 26.8±4.5 mg for the DX3Puroβ₆ tumors. At 4 h p.i. tumor contrast was clearly visible. As radioactivity levels dropped in the blood over time, tumor contrast increased throughout the 12 h and 24 h time points, with the highest contrast observed at 24 h p.i and confirmed by biodistribution (DX3Puroβ₆: 4.63±0.18% ID/g, DX3Puro: 3.92±0.30% ID/g). Although a direct comparison cannot be made to the [¹⁸F]-SFB-labeled diabody and cys-diabodies due to the differences in isotope and time points measured, tumor uptake continued to increase and was highest for the site-specifically labeled [⁶⁴Cu]-NOTA-αᵥβ₆ Cys-diabody at 24 h. Clearance of the [⁶⁴Cu]-NOTA-αᵥβ₆ Cys-diabody was observed through the renal and hepatobiliary pathways with prominent uptake observed in the PET images for both the kidneys and the liver. Ex vivo biodistribution confirmed uptake of the [⁶⁴Cu]-NOTA-αᵥβ₆ Cys-diabody in the kidneys (9.48±2.29% ID/g) and liver (11.29±2.38% ID/g) at 24 h p.i.

D. Immunohistochemistry (IHC) and Autoradiography of [¹⁸F]-FB-αᵥβ₆ Diabody

Correlation of the in vivo PET and ex vivo biodistribution data was also confirmed by autoradiography and IHC of the sliced tumors. Mice were euthanized at 6 h p.i. followed by removal and slicing of tumors (20 μM thickness) using a cryotome at −20° C. Tumor slices were exposed were overnight to a phosphor imaging screen and the images were developed at a 50 μM resolution the following day. The autoradiography image revealed increased signal (darker contrast) for the DX3Puroβ₆ tumor slices versus those obtained from the DX3Puro tumors.

Immunohistochemical staining of the excised tumors for the αᵥβ₆ integrin revealed intense localization of this cell surface receptor, particularly around the outer edges of the tumors. The distribution of the anti-αᵥβ₆ diabody was much more dispersed throughout the tumors when compared to the staining for αᵥβ₆, which was localized to the edges.

E. Immunohistochemistry (IHC) and Autoradiography of [¹⁸F]-FB-αᵥβ₆ Cys-Diabody

Autoradiography and immunohistochemical staining was used to evaluate tumor localization of the [¹⁸F]-FB-αᵥβ₆ Cys-diabody. At 6 h p.i., mice were euthanized, tumors were excised and sliced (20 μM for autoradiography, 5 μM for IHC) using a cryotome at −20° C. Following autoradiography, the DX3Puroβ₆ tumor slices had noticeably darker contrast than the DX3Puro tumor slices indicating increased radiotracer uptake in the DX3Puroβ₆ tumors. These results supported the previous observations from ex vivo biodistribution in which the tumor uptake was higher for the DX3Puroβ₆ tumors.

Localization of the anti-αᵥβ₆ Cys-diabody by IHC staining revealed results consistent to what was observed with ex vivo biodistribution. The staining of the anti-αᵥβ₆ Cys-diabody was more intense in the DX3Puroβ₆ tumors compared to the DX3Puro tumors. When separate slices of each tumor were stained for the αᵥβ₆ integrin, there was a marked difference in intensity. The DX3Puroβ₆ slices had very intense staining around the periphery whereas the DX3Puro slices showed very faint, if any staining of αᵥβ₆.

### Conclusion

This example illustrates the use of three radiolabeled diabodies (e.g., [¹⁸F]-FB-αᵥβ₆ diabody, [¹⁸F]-FB-αᵥβ₆ Cys-diabody, and [⁶⁴Cu]-NOTA-αᵥβ₆ diabody) for imaging αᵥβ₆ integrin expression in vivo with PET. These diabodies generated rapid tumor contrast which is required to conduct same-day molecular imaging.

### Example 4

### Methods for Site-Specific [¹⁸F] Radiolabeling of Anti-αᵥβ₆ Cys Diabodies Using [¹⁸F]-FBEM

This example illustrates the incorporation of [¹⁸F] at site-specific sites on anti-αᵥβ₆ diabodies and anti-αᵥβ₆ Cys-diabodies.

The thiol reactive N-[2-(4-18F-fluorobenzamido)ethyl]maleimide ([¹⁸F]-FBEM) was employed to site-specifically radiolabel the anti-αᵥβ₆ Cys-diabody following reduction of the engineered disulfide bond. This prosthetic group was synthesized following the automated production of [¹⁸F]-SFB following a one step heating reaction. The synthesis and purification of [¹⁸F]-FBEM and the site-specific radiolabeling of the anti-αᵥβ₆ Cys-diabody was performed according to the methods outlined below.

Dried [¹⁸F]-SFB was redissolved in a solution of anhydrous acetonitrile (500 μL) containing the amino-ethyl-maleimide precursor (1.75 mg, Matrix Scientific). Diisopropylethylamine (DIPEA, 25 μL) was added to the reaction which was heated to 40° C. for 20 mins. The reaction was dried under nitrogen and redissolved in 0.05% TFA/water and acetonitrile (1:1 v/v). C rude [¹⁸F]-FBEM was purified using semi-preparative HPLC as described for [¹⁸F]-SFB and trapped onto a C18 Sep-Pak Plus cartridge. Purified [¹⁸F]-FBEM was eluted with dichloromethane (2 mL) and dried under a stream of nitrogen. Synthesis of [¹⁸F]-FBEM was conducted within 66 mins after receiving [¹⁸F]-SFB, and HPLC purification and formulation required an additional 59 mins. Synthesis of [¹⁸F]-FBEM was performed with a decay-corrected RCY of 34% before HPLC purification. HPLC purification yielded a final product purity >98%.

The anti-αᵥβ₆ Cys-diabody (1 mg/mL, 100 μL) was buffer-exchanged into 0.1 M NaOAc, pH 7 using a BioSpin-30 column (BioRad). Reduction of the disulfide bond was performed using TCEP (25 mM final concentration) at room temperature for 30 mins with occasional mixing. The reduced Cys-diabody (87 μg) was added to dried [¹⁸F]-FBEM (1.02 mCi). The reaction was incubated at room temperature for 45 mins. A BioSpin-30 column (40 kDa MWCO, BioRad) pre-equilibrated with PBS was used to separate unreacted [¹⁸F]-FBEM from the [¹⁸F]-FBEM-αᵥβ₆ Cys-diabody. The RCY and RCP for the [¹⁸F]-FBEM-αᵥβ₆ Cys-diabody were determined as described for the [¹⁸F]-SFB-αᵥβ₆ Cys-diabody.

### Example 5

### Methods for Site-Specific [¹⁸F] Radiolabeling of Anti-αᵥβ₆ Cys Diabodies Using [¹⁸F]-TCO

This example illustrates incorporating of [¹⁸F] at site-specific sites on anti-αᵥβ₆ Cys diabodies using the tetrazine-transcyclooctene (TTCO) ligation based on the inverse electron demand Diels-Alder reaction. The premise of this technique relies on the extremely fast, bioorthogonal reaction kinetics between the tetrazine and the most highly reactive dienophile, the transcyclooctene. The reaction kinetics proceed at rates much higher than any catalyzed click reactions such as the 1,3-dipolar cycloaddition and even faster than strain-promoted cycloadditions between azides and strained alkynes. The lack of catalyst eliminates the need to remove toxic constituents, but more importantly enables this reaction to be performed under biological conditions with the only byproduct being N₂. Such a reaction can be performed with the αᵥβ₆ Cys-diabody in one of two methods: 1) the conjugation of the TCO to the Cys-diabody after reduction of the disulfide bond, followed by the TTCO click reaction with the radiofluorinated tetrazine, or 2) the Cys-diabody is first be site-specifically conjugated with the tetrazine moiety followed by the TTCO click reaction with the radiofluorinated TCO.

Provided herein is a description of the method involving the radiofluorination of the transcyclooctene. [¹⁸F]-fluoride was obtained from PETNET (68.7 mCi) onto a trap and release column (pre-equilibrated with 2 mL of H₂O, dried with air). The trap and release column was eluted with 4-[(1,1-dimethylethoxy)carbonyl]-N,N,N-trimethyl,1,1,1-trifluoromethanesulfonate (1:1) (TBAB) into a conical vial (5 mL). Eluted [¹⁸F]-fluoride was azeotropically dried with three fractions of MeCN (1 mL each) at 95° C. under a stream of nitrogen. TCO-nosylate precursor (2 mg, Fox Laboratory, University of Delaware) in MeCn (500 μL) was added to the dried [¹⁸F]-fluoride and the reaction was heated to 75° C. for 15 mins. To acidify the crude reaction, 500 μL of HPLC solvent A (0.05% TFA in H2O) was added and the reaction was purified by semi-prep HPLC. The HPLC-purified product was solvent-exchanged using a C-18 SepPak cartridge (Waters), washed with H2O 2O (5 mL) and dried with air. The cartridge was then eluted with DCM (1-2 mL). Overall synthesis of [¹⁸F]-TCO was accomplished in 90 mins including semi-prep HPLC purification. The RCY was 11.6% without decay correction.

Next, the tetrazine-maleimide was conjugated to the reduced anti-αᵥβ₆ Cys-diabody. Briefly, the anti-αᵥβ₆ Cys-diabody (100 μL, 50.6 μM) was reduced with tris(2-carboxyethyl)phosphine (TCEP) Bond Breaker™ (25 mM, Pierce). The reaction was mixed and allowed to proceed at 4° C. for 30 mins. Excess TCEP was removed using a Biospin-30 spin column (40 kDa MWCO, BioRad) pre-equilibrated with PBS, 0.5M NaCl, pH 7. Tetrazine-maleimide (50 molar excess, 252 nmol, Jena Bioscience) was dissolved in DMSO (6.3 μL) and added directly to the reduced Cys-diabodies. The conjugation reaction proceeded for 2 hrs at 4° C. with occasional shaking. Excess unconjugated tetrazine-maleimide was removed with a Biospin-30 spin column. Eluted tetrazine-conjugated Cys-diabody was stored at 4° C. Confirmation of the site-specific conjugation of the tetrazine-maleimide to the anti-αᵥβ₆ Cys-diabody was performed by using electrospray ionization.

To test the feasibility of site-specific radiolabeling using the [¹⁸F]-TTCO reaction mechanism, we conducted radiolabeling experiments with the tetrazine-αᵥβ₆ Cys-diabody. The tetrazine-αᵥβ₆ Cys-diabody (2.1 mg/mL, 63 μg) in storage buffer (0.1M HEPES, 0.5M NaCl, 10% glycerol, pH 7) was added to dried [18F]-TCO (650 μCi) and incubated at 37° C. for 20 mins. Unreacted [¹⁸F]-TCO was removed via spin-column purification (Biospin-30, BioRad), and the eluted [¹⁸F]-TTCO-αᵥβ₆ Cys-diabody was injected onto size-exclusion HPLC (eluent: 0.1M phosphate buffer, pH 6.8, flow rate: 0.5 mL/min) to verify final radiochemical purity. The RCY was determined by dividing the eluted radioactivity present as the [¹⁸F]-TTCO-αᵥβ₆ Cys-diabody by the initial radioactivity applied to the spin column. RCY was approximately 4.7%, non-decay corrected and RCP appeared to be greater than 95%.

### Example 6

### Methods for Generating Anti-αᵥβ₆ Integrin Diabodies and Disulfide-Stabilized Anti-αᵥβ₆ Integrin Diabodies Containing Unnatural Amino Acids

This example illustrates the incorporation of unnatural amino acids into the coding region of anti-αᵥβ₆ diabodies and anti-αᵥβ₆ Cys-diabodies specific for αᵥβ₆ integrin.

To expand the genetic code to incorporate unnatural amino acids (UAAs) into the biochemical machinery, several new components must be created with strict requirements. First, a UAA must be synthesized that is metabolically stable. The UAA cannot be a substrate for endogenous aminoacyl-tRNA synthetases (aaRSs) and it must be tolerated by the ribosome during translation. Second, a unique aaRS/tRNA pair must be created that only recognizes the new UAA. This pair must also be orthogonal to each other with respect to endogenous aaRS/tRNA pairs. Finally, a unique mRNA codon must be created that only recognizes the new tRNA and encodes only the UAA. The advantage of using this technique is twofold. By altering the genetic machinery, precise control over selective introduction of the UAA can be obtained. Thus, regions of the diabody fragment that encode the CDRs involved in antigen recognition can be avoided; reducing or possibly eliminating any deleterious effects on affinity. Furthermore, site-specific radiolabeling of the diabody can be accomplished via rapid click chemistry methodologies such as the TTCO ligation described above. These UAAs make it possible to generate diabodies with novel properties for imaging αᵥβ₆-positive tumors with PET.

Several groups have generated orthogonal aaRS/tRNA pairs to incorporate various unnatural phenylalanine, tyrosine, and lysine derivatives into recombinant proteins. In efforts to perturb the natural diabody sequence as little as possible, the pyrrolysine aaRS/tRNA pairs developed from the single-cell archaea Methanocarcina barkeri (Mb) were used in order to replace natural lysines with unnatural lysine derivatives. In Methanocarcina barkeri, the tRNA synthetase MbPyrlRS and its cognate amber suppressor MbtRNA_{CUA} direct the incorporation of pyrrolysine. The MbtRNA_{CUA} (encoded by the MbpylT gene) naturally contains a CUA anticodon that specifically suppresses the amber codon TAG during the translation of pyrrolysine. It has been demonstrated that MbPyrlRS forms a naturally orthogonal pair with MbtRNA_{CUA} in E. coli and that MbtRNA_{CUA} is not a substrate for endogenous aaRSs. The naturally orthogonal MbPyrlRS/MbtRNA_{CUA} pair may therefore provide the basis to introduce pyrrolysine derivatives into the anti-αᵥβ₆ diabody.

The pCMV-MbPyl-MamOpt and pCMV-MmPyl-MamOpt plasmids and N₃-lysine were used. The plasmids encode the wild-type MbPyl synthetase and MmPyl synthetase, respectively, which are driven by a CMV promoter. They also include the wild-type MmPyl tRNA driven by the U6 promoter. The vector allows for simultaneous expression of the MbPyrlRS/MbtRNACUA pair in mammalian expression systems and contains an ampicillin and hygromycin resistance genes for screening. Variants of the pSecTag plasmid expressing the anti-αᵥβ₆ diabody with the TAG codon located at position 228 (pSecTag-B6DiaR-G2S-228TAG) and another version with the TAG codon located at position 232 (pSecTag-B6DiaR-G2S-232TAG) were generated. These locations for introduction of the TAG codon were chosen for two reasons. First, the C-terminal location is distant from the CDR regions and introduction of a lysine derivative here is least likely to interfere with antigen recognition. Secondly, the presence of natural lysines at these locations should reduce any deleterious effects caused by adding an unnatural lysine derivative.

To incorporate the N₃-lysine into the anti-αᵥβ₆ diabody sequence, a transfection array was created in which we cotransfected various combinations of the pCMV-MbPyl-MamOpt, pCMV-MmPyl-MamOpt, pSecTag-B6DiaR-G2S-228TAG, and pSecTag-B6DiaR-G2S-232TAG plasmids into mammalian 293-F cells (2 mL cultures) while feeding the cells increasing amounts of N₃-lysine. This was conducted to determine what effects the location of the TAG codon, the synthetase used, and the amounts of N₃-lysine present have on UAA incorporation. Supernatants from the array were harvested and electrophoresed on a 4-20% precast gel followed by western blotting. The membrane was probed with the anti-Penta His-HRP antibody to detect the presence of the UAA-modified diabodies. This method was chosen because detection of the UAA-modified diabody can only occur if translation proceeds through the location of the TAG codon, confirming insertion of the N3-lysine and translating the hexahistadine tag at the c-terminus. If insertion of the N3-lysine does not occur, translation of the diabody will stop at the TAG codon and the diabody will be truncated before the hexahistidine tag can be translated.

The results showed that an N₃-lysine-containing diabody was generated at low levels. Regardless of the location of the TAG codon, only transfections containing pCMV-MbPyl-MamOpt plasmid yielded N₃-lysine-modified diabody that was detectable by western blot. Those transfections that contained the pCMV-MmPyl-MamOpt plasmid did not yield detectable diabody that had been modified with the N₃-lysine. It appears that increasing the amounts of N₃-lysine in the growth media past 1 mM did not significantly affect the incorporation efficiency.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Informal Sequence Listing

SEQ ID NO: 1
Human V_{H} chain of α_{V}β₆ integrin antibody
EVQLVESGGGLVQPGGSLRL SCAASGFTFSRYVMSWVRQA
PGKGLEWVASISSGGRMYYP DTVKGRFTISRDNAKNSLYL
QMNSLRAEDTAVYYCARGSI YDGYYVFPYWGQGTLVTVSS
SEQ ID NO: 2
Human V_{L} chain of α_{V}β₆ integrin antibody
EIVLTQSPATLSLSPGERAT LSCSASSSVSSSYLYWYQQK
PGQAPRLLIYSTSNLASGIP ARFSGSGSGTDFTLTISSLE
PEDFAVYYCHQWSTYPPTFG GGTKVEIK
SEQ ID NO: 3
Nucleic acid sequence of human V_{H} chain of αᵥβ₆
integrin antibody
gag gtg cag ctg gtg gag agc ggc ggc ggc ctg gtg
cag ccc ggc ggc agc ctg agg ctg agc tgc gcc gcc
agc ggc ttc acc ttc agc cgc tac gtg atg agc tgg
gtg cgc cag gcc ccc ggc aag ggc ctg gag tgg gtg
gcc agc atc agc agc gga ggc cgc atg tact ac ccc
gac acc gtg aag ggc cgc ttc acc atc agc cgc gac
aac gcc aag aac agc ctg tac ctg cag atg aac agc
ctg cgc gcc gag gac acc gcc gtg tact ac tgc gcc
cgt ggc agc atc tac gac ggc tact ac gtg ttc ccc
tac tgg ggc cag ggc acc ctg gtg acc gtg agc tc
SEQ ID NO: 4
Nucleic acid sequence of human V_{L} chain of αᵥβ₆
integrin antibody
gag atc gtg ctg acc cag agc ccc gcc acc ctg agc
ctg agc ccc ggc gag agg gcc acc ctg agc tgc agc
gcc agc agc agc gtg agc agc gac tac ctg tac tgg
tac cag cag aag ccc ggc cag gcc ccc agg ctg ctg
atc tac agc acc agc aac ctg gcc agc ggc atc ccc
gcc cgc ttc agc ggc agc ggc acc gac ttc acc ctg
acc atc agc agc ctg gag ccc gag gac ttc gcc gtg
tact ac tgc cac cag tgg agc acct ac ccc ccc acc
ttc ggc ggc ggc acc aag gtg gag atc aag

1. An isolated multivalent antibody fragment that specifically binds αVβ6 integrin comprising two or more single-chain Fv (scFv) molecules that associate with each other, wherein each scFv molecule independently comprises the following structure:
(a) a light chain variable (VL) region and a heavy chain variable (VH) region of an antibody that specifically binds αVβ6 integrin; and
(b) a peptide linker between the VL region and the VH region.

2. The antibody fragment of claim 1, wherein the antibody fragment comprises at least one unnatural amino acid.

3. The antibody fragment of claim 1, wherein a cysteine or thiol residue has been inserted at the C-terminus of each scFv molecule.

4. The antibody fragment of claim 1, wherein the VL region and the VH region are derived from the humanized hu6.3G9 antibody.

5. The antibody fragment of claim 1, wherein the VL region comprises a polypeptide having the amino acid sequence of SEQ ID NO:1.

6. The antibody fragment of claim 1, wherein the VH region comprises a polypeptide having the amino acid sequence of SEQ ID NO:2.

7. The antibody fragment of claim 1, wherein the peptide linker is less than about 15 amino acids in length.

8. The antibody fragment of claim 7, wherein the peptide linker is from 0 to about 8 amino acids in length.

9. The antibody fragment of claim 1, wherein the antibody fragment is selected from the group consisting of a diabody, a minibody, a triabody, and a tetrabody.

10. The antibody fragment of claim 1, wherein an imaging agent or a therapeutic agent is conjugated to the antibody fragment.

11. The antibody fragment of claim 10, wherein the imaging agent or the therapeutic agent is conjugated to a predetermined site in the antibody fragment.

12. The antibody fragment of claim 11, wherein the predetermined site is an unnatural amino acid in the antibody fragment.

13. The antibody fragment of claim 11, wherein the predetermined site is a cysteine or thiol residue that has been inserted at the C-terminus of each scFv molecule.

14. The antibody fragment of claim 10, wherein the imaging agent or the therapeutic agent is a radionuclide.

15. The antibody fragment of claim 14, wherein the radionuclide is selected from the group consisting of 11C, 13N, 15O, 18F, 19F, 61Cu, 62Cu, 64Cu, 67Cu, 68Ga, 111In, 124I, 125I, and 131I.

16. A composition comprising an antibody fragment of claim 1 and a pharmaceutically acceptable carrier.

17. A kit for imaging or therapy, the kit comprising:
(a) an antibody fragment of claim 1; and
(b) instructions for use thereof for imaging or therapy.

18. A method of in vivo imaging of a target tissue in a subject, the method comprising:
(a) administering to the subject in need of such imaging, an antibody fragment of claim 1, wherein an imaging agent is conjugated to the antibody fragment; and
(b) detecting the antibody fragment to determine where the antibody fragment is concentrated in the subject.

19. The method of claim 18, wherein the target tissue is a cancerous tissue or an organ.

20. The method of claim 19, wherein the cancerous tissue is associated with pancreatic cancer, breast cancer, colorectal cancer, prostate cancer, or oral squamous cell carcinoma.

21. The method of claim 18, wherein the imaging agent is a radionuclide, and wherein radiation from the radionuclide is used to determine where the antibody fragment is concentrated in the subject.

22. The method of claim 18, wherein the radionuclide is selected from the group consisting of 11C, 13N, 15O, 18F, 19F, 61Cu, 62Cu, 64Cu, 67Cu, 68Ga, 111In, 124I, 125I, and 131I.

23. The method of claim 18, wherein the antibody fragment is detected by Magnetic Resonance Imaging (MM), Magnetic Resonance Spectroscopy (MRS), Single Photon Emission Computerized Tomography (SPECT), Positron Emission Tomography (PET), or optical imaging.

24. The method of claim 18, wherein the subject has an αvβ6 integrin-mediated disease or disorder.

25. A method of treating an αvβ6 integrin-mediated disease or disorder in a subject in need thereof, the method comprising:
administering to the subject a therapeutically effective amount of an antibody fragment of claim 1, wherein a therapeutic agent is conjugated to the antibody fragment.

26. The method of claim 25, wherein the therapeutic agent is a radionuclide.

27. The method of claim 26, wherein the radionuclide is selected from the group consisting of 11C, 13N, 15O, 18F, 19F, 61Cu, 62Cu, 64Cu, 67Cu, 68Ga, 111In, 124I, 125I, and 131I.

28. The method of claim 25, wherein the αvβ6 integrin-mediated disease or disorder is pancreatic cancer, breast cancer, colorectal cancer, prostate cancer, or oral squamous cell carcinoma.

29. The method of claim 25, wherein the therapeutically effective amount of the antibody fragment is an amount sufficient to target delivery of the therapeutic agent to a cell expressing the αvβ6 integrin.


SEQUENCE LISTING (9 sequences)

SEQ ID NO: 1 - protein, 120 aa, Artificial Sequence
  synthetic humanized hu6.3G9 anti-alpha-vbeta-6 integrin antibody variable heavy chain (V-H)
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSS

SEQ ID NO: 2 - protein, 108 aa, Artificial Sequence
  synthetic humanized hu6.3G9 anti-alpha-vbeta-6 integrin antibody variable light chain (V-L)
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIK

SEQ ID NO: 3 - DNA, 360 bp, Artificial Sequence
  synthetic humanized hu6.3G9 anti-alpha-vbeta-6 integrin antibody variable heavy chain (V-H)
gaggtgcagctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacaacgccaagaacagcctgtacctgcagatgaacagcctgcgcgccgaggacaccgccgtgtactactgcgcccgtggcagcatctacgacggctactacgtgttcccctactggggccagggcaccctggtgaccgtgagctcc

SEQ ID NO: 4 - DNA, 324 bp, Artificial Sequence
  synthetic humanized hu6.3G9 anti-alpha-vbeta-6 integrin antibody variable light chain (V-L)
gagatcgtgctgacccagagccccgccaccctgagcctgagccccggcgagagggccaccctgagctgcagcgccagcagcagcgtgagcagcagctacctgtactggtaccagcagaagcccggccaggcccccaggctgctgatctacagcaccagcaacctggccagcggcatccccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaag

SEQ ID NO: 5 - protein, 5 aa, Artificial Sequence
  synthetic flexible peptide linker
GGGGS

SEQ ID NO: 6 - protein, 6 aa, Artificial Sequence
  synthetic 6xHis, histidine tag, purification tag, hexahistidine tag
HHHHHH

SEQ ID NO: 7 - protein, 5 aa, Artificial Sequence
  synthetic penta-His
HHHHH

SEQ ID NO: 8 - protein, 5 aa, Artificial Sequence
  synthetic linker separating V-H and V-L domains
GGGGS

SEQ ID NO: 9 - DNA, 318 bp, Artificial Sequence
  synthetic humanized hu6.3G9 anti-alpha-vbeta-6 integrin antibody variable light chain (V-L)
gagatcgtgctgacccagagccccgccaccctgagcctgagccccggcgagagggccaccctgagctgcagcgccagcagcagcgtgagcagcgactacctgtactggtaccagcagaagcccggccaggcccccaggctgctgatctacagcaccagcaacctggccagcggcatccccgcccgcttcagcggcagcggcaccgacttcaccctgaccatcagcagcctggagcccgaggacttcgccgtgtactactgccaccagtggagcacctacccccccaccttcggcggcggcaccaaggtggagatcaag